(19) United States
(12) Patent Application Publication
(10) Pub. No.: US 2025/0107973 A1
(43) Pub. Date: Apr. 3, 2025

(54) SILICA CAPSULES

(71) Applicant: The Procter & Gamble Company, Cincinnati, OH (US)

(72) Inventors: Andre Martim BARROS, Woluwe Saint Etienne (BE); Mariana B. T. CARDOSO, Brussels (BE); Jane Ellen GODLEWSKI, Loveland, OH (US); Loic Martin Christian VANDEREST, Etterbeek (BE); Nadia DELLA CORTE, Brussels (BE); Laura ORLANDINI, Le Mont-Sur-Lausanne (CH)

(21) Appl. No.: 18/771,315

(22) Filed: Jul. 12, 2024

Related U.S. Application Data
(60) Provisional application No. 63/514,369, filed on Jul. 19, 2023.

(51) Int. Cl. A61K 8/11; A61Q 13/00

(52) U.S. Cl. CPC A61K 8/11; A61Q 13/00; A61K 2800/56; A61K 2800/805

(57) ABSTRACT

A population of capsules comprising: a core comprising a benefit agent and a shell surrounding the core; wherein the shell surrounding the core comprises a substantially inorganic first shell; wherein the substantially inorganic first shell comprises a condensed layer comprising a condensation product of a mixture of precursors; and wherein the mixture of precursors comprises a silica precursor and a crosslinking metal-oxide precursor.

### FIELD OF THE INVENTION

The disclosure relates to capsules and methods of making capsules for the transfer and triggered release of benefit agents.

### BACKGROUND OF THE INVENTION

The formation of core-shell capsules based on silica chemistry has been of particular interest in recent years. There are publications on different strategies to obtain core-shell silica capsules, but most of these capsules do not possess adequate barrier properties, allowing premature leakage of active into surfactant and solvent rich consumer good formulations. Some strategies to prevent leakage include applying coatings to the capsule shell to increase the shell thickness; however this approach has limitations as the main drivers of low porosity of a material comes from the scaffold of the initial shell structure, rather than any subsequent coatings. This becomes particularly important in solvent-based consumer goods, as the small solvent molecules are capable of penetrating deeper through solid materials than larger surfactant molecules.

It is known in the art that by doping a silica matrix with titania, zirconia, and other metal oxides that the resulting network is less porous. This is trivial to achieve when working in a homogeneous system where all components are homogeneous together at the start and where the subsequent solid material fills the entire reaction vessel (for example when making xerogels and aerogels). However, in the case of interfacial polymerization and encapsulation in particular, the added challenge is to ensure that the reagents react primarily at the oil/water (O/W) interface, but also to ensure that they react in similar time scales so as to avoid the formation of concentrated zones of one metal-oxide in another-metal oxide. To observe the reduced porosity a co-polymer of the two or more metal-oxides must be formed.

There is a continued need to further improve the barrier properties of silica capsules.

To overcome these challenges, it has been found that using specific precursors of silica and a second metal-oxide, a homogeneously blended mixed oxide material can be obtained during encapsulation to form a lower porosity shell surrounding a core material.

### SUMMARY OF THE INVENTION

In accordance with embodiments, a population of core/shell capsules contain either an oil-based core or a water-based core, and a shell surrounding the core. In embodiments, the shell can include a first shell component and a second shell component, wherein the second shell component is surrounding the first shell component. In embodiments, the first shell component can include a condensed layer and a nanoparticle layer, wherein the condensed layer is disposed between the core and the nanoparticle layer. In embodiments, the condensed layer can include a condensation product of a precursor mixture.

In embodiments, the precursor mixture comprises a silica precursor and a crosslinking metal-oxide precursor. The combination of precursor mixture can be selected such that its components have similar reaction rates.

### DETAILED DESCRIPTION OF THE INVENTION

The present invention discloses capsules based on silica shell with improved barrier properties by a careful selection of precursor mixture which can overcome the challenges facing the current state of the art. This precursor mixture hydrolyzes and condenses at an oil/water interface to create a mixed-oxide shell surrounding a dispersed droplet. The mixed-oxide shell primarily composes silica, with small fractions of a crosslinking metal-oxide that promote the crosslinking of silica and lower the porosity of the subsequently formed shell, thereby greatly improving the barrier properties of the capsules. The precursor mixture comprises silica precursors and a crosslinking metal-oxide precursor.

As used herein, when the term “about” modifies a particular value, the term refers to a range equal to the particular value, plus or minus twenty percent (±20%). For any of the embodiments disclosed herein, any disclosure of a particular value, can, in various alternate embodiments, also be understood as a disclosure of a range equal to about that particular value (i.e. ±20%).

As used herein, when the term “substantially” modifies a particular value, unless otherwise specifically defined in the specification, the term refers to a range equal to the particular value, plus or minus ten percent (±10%). For any of the embodiments disclosed herein, any disclosure of a particular value, can, in various alternate embodiments, also be understood as a disclosure of a range equal to approximately that particular value (i.e. ±10%).

As used herein, the word “or” when used as a connector of two or more elements is meant to include the elements individually and in combination; for example X or Y, means X or Y or both.

As used herein, the articles “a” and “an” are understood to mean one or more of the material that is claimed or described.

It has been found that to obtain capsules of the present invention by interfacial polymerization of the precursor mixture resulting in the formation of a mixed-oxide shell, the hydrolysis reaction kinetics of the crosslinking metal-oxide precursor must be greatly reduced to confer the desired barrier properties to the capsules.

Without wishing to be bound by theory, it is believed that if the hydrolysis and condensation reaction rate of the two precursors (i.e. silica and a second metal oxide) are too different from one another, the resulting shell contains too little second metal-oxide condensation products. A difference in the hydrolysis reaction rate can also lead to a mixed-oxide shell which contains spotty domains of condensation products of the second metal-oxide. To obtain a low porosity shell and therefore a capsule with improved barrier properties, it is important to ensure the homogeneous incorporation of the two or more materials that will form the shell.

Without wishing to further be bound by theory, it is believed that when the crosslinking metal-oxide precursor hydrolyzes too quickly, it becomes dispersed in the aqueous phase and will no longer form a mixed-oxide shell when used together with a silica precursor. The resulting shell in this scenario will not contain a homogeneous distribution of the crosslinking metal-oxide in the shell. This homogeneous distribution of the crosslinking metal-oxide is important to promote the crosslinking of the shell, which greatly reduces the porosity of the shell and greatly improves the barrier properties of the shell, in particular against small organic solvent molecules.

Further, when the hydrolysis and condensation of the silica precursor and the crosslinking metal-oxide precursor are similar, the resulting shell will be a mixed-oxide shell where the crosslinking metal-oxide is homogeneously distributed in the silica network. Since the relative reactivity of the components comprising the precursor mixture is important to control, it is by a careful selection of the silica precursor and of the crosslinking metal-oxide precursor that capsules of the present invention can be obtained.

Furthermore, it has been found that only a small quantity of the crosslinking metal-oxide is needed to be embedded in the silica network of the shell in order to obtain strong crosslinking. If this value is too low or too high, this will negatively impact the barrier properties of the shell.

Without wishing to be bound by theory, if one were to simply apply a coating of the crosslinking metal-oxide onto the silica shell, this would not result in the formation of capsules of the present invention for at least two reasons: (1) the coating would not be homogeneously distributed throughout the network of the silica shell, which is a desired condition to obtain a more crosslinked shell; and (2) the crosslinking of the shell occurs when the curing of the shell is still proceeding, since the bonds of the shell are still labile. The crosslinking metal-oxide will be able to reduce the porosity only when its precursor is co-polymerizing with the silica precursor.

In fact point (2) from above is the reason why a homogeneously distributed crosslinking metal-oxide can be obtained, since it is a consequence of the co-polymerization of the crosslinking metal-oxide precursor and the silica precursor.

In accordance with embodiments of the present invention, inorganic capsules having a core surrounded by a shell are provided. The core can include one or more benefit agents. In various embodiments, the shell can include a first shell component and optionally a second shell component that substantially surrounds the first shell component. In embodiments, the first shell component can include a condensed layer formed from the condensation product of a precursor. As described in detail below, the precursor can include a precursor mixture. In embodiments, the first shell component can include a nanoparticle layer. In embodiments, the second shell component can include inorganic materials.

Capsules of the present invention are defined as comprising a substantially inorganic shell. By substantially inorganic it is meant that the first shell component can comprise up to 10 wt %, preferably 9 wt %, preferably 8 wt %, preferably 7 wt %, preferably 6 wt %, preferably 5 wt %, preferably 4 wt %, preferably 3 wt %, preferably 2 wt % preferably 1 wt % of organic content as defined later in the organic content calculation and later in the description. In a particular preferred embodiment the organic content is 0% and the capsule shell is fully inorganic.

Furthermore, the condensed layer can be made from a condensation product of at least one silica precursor and a crosslinking metal-oxide precursor. While it is possible to obtain a silica shell capsule from the usage of a silica precursor alone, the combined usage of silica precursors and crosslinking metal-oxide precursors leads to capsules with superior barrier properties.

In the present invention, capsules may be formed by first admixing a hydrophobic material with a precursor mixture. Said precursor/oil mixture is then either used as a dispersed phase or as a continuous phase in conjunction with a water phase, where in the former case an oil-in-water emulsion is formed and in the latter a water-in-oil emulsion is formed once the two phases are mixed and homogenized via methods that are known to the person skilled in the art.

The precursor mixture will start undergoing a hydrolysis reaction with water at the oil/water interface to obtain a partially hydrolyzed precursor mixture with hydroxy groups. The partially hydrolyzed precursor mixtures are then able to react with each other to form a mixed oxide shell.

The silica precursor comprises molecules and polymers that are substantially water immiscible (i.e. oil-soluble) and that are fully hydrolysable, such that upon undergoing hydrolysis and condensation reactions they will form silica or SiO₂. By fully hydrolysable it is meant that there are no Silicon-carbon bonds in the precursor, or other similarly unhydrolyzable functional groups. Non-limiting examples of silica precursors are silicon alkoxides, acetoxy-silanes, silicon-halides and oligomers obtainable from their polymerization.

The crosslinking metal-oxide precursor comprises molecules and polymers that are substantially water immiscible (i.e. oil-soluble) and that are fully hydrolysable, such that upon undergoing hydrolysis and condensation reactions they will form a crosslinking metal-oxide. Examples of crosslinking metal-oxide precursors and crosslinking metal-oxides are discussed below.

### Shell

The capsules of the present invention comprise a shell surrounding a core, wherein the shell comprises a substantially inorganic first shell component. The first shell component can include a condensed layer and a nanoparticle layer. The shell can further include a second shell component.

### Condensed Layer

The condensed layer comprises a condensation product of a precursor mixture, which comprises at least one silica precursors and one crosslinking metal-oxide precursor.

The silica precursors comprise oil-soluble and fully hydrolysable precursors of Silica or SiO₂. The oil-soluble and fully hydrolysable precursors of Silica or SiO₂ comprises at least one of the compounds of formula (I) or formula (II):

SiY₄ Formula (I)
where each Y is independently selected from
OH, —OR², halo,

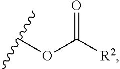

NH₂, —NHR², —N(R²)₂, and

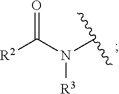

wherein R² is a C1 to C20 alkyl, C1 to C20 alkylene, C6 to C22 aryl, or a 5-12 membered heteroaryl comprising from 1 to 3 ring heteroatoms selected from O, N, and S;
R³ is a H, C1 to C20 alkyl, C1 to C20 alkylene, C6 to C22 aryl, or a 5-12 membered heteroaryl comprising from 1 to 3 ring heteroatoms selected from O, N, and S.

In embodiments, the compound of formula (I) is a silicon alkoxide. The silicon alkoxide can be for example tetramethoxy silane (TMOS), tetraethoxy silane (TEOS), tetraisopropoxy silane or tetrabutoxysilane (TBOS).

(SiO_{z}Yₙ)_{w} Formula (II)
where z is from 0.5 to 1.6;
each Y is independently selected from:
OH, —OR², halo,

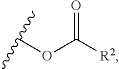

NH₂, —NHR², —N(R²)₂, and

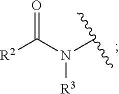

wherein R² is a C1 to C20 alkyl, C1 to C20 alkylene, C6 to C22 aryl, or a 5-12 membered heteroaryl comprising from 1 to 3 ring heteroatoms selected from O, N, and S;
R³ is a H, C1 to C20 alkyl, C1 to C20 alkylene, C6 to C22 aryl, or a 5-12 membered heteroaryl comprising from 1 to 3 ring heteroatoms selected from O, N, and S;
n is from 0.7 to 4; and
w is from 2 to 2000.

In embodiments, Y is an alkoxy group. In embodiments, the alkoxy group includes methoxy, ethoxy, isopropoxy and butoxy groups.

In embodiments, the silica precursor can include polyalkoxysilane (PAOS).

In embodiments, the compound of formula (II) can have a Polystyrene equivalent Weight Average Molecular Weight (Mw) of from about 100 Da to about 300,000 Da. In embodiments, the Mw can be from about 100 Da to about 100,000 Da, or from about 100 Da to about 90,000 Da, or from about 100 Da to about 80,000 Da, or from about 100 Da to about 70,000 Da, or from about 100 Da to about 60,000 about Da, or from about 200 Da to about 60,000 Da, or from about 300 Da to about 60,000 Da, or from about 400 Da to about 60,000 Da, or from about 500 Da to about 60,000 Da, or from about 600 Da to about 60,000 Da, or from about 700 Da to about 60,000 Da, or from about 700 Da to about 30,000 Da, or from about 800 Da to about 30,000 Da, or from about 900 Da to about 30,000 Da, or from about 1000 Da to about 30,000 Da, or from about 1500 Da to about 30,000 Da.

In embodiments, the compound of formula (II) can have a molecular weight polydispersity index of about 1 to about 50. In embodiments, the molecular weight polydispersity index can be from about 1 to about 45, or about 1 to about 40, or about 1 to about 30 or about 1 to about 25, or about 1 to about 20, or about 1 to about 15, or about 1 to about 10, or about 1 to about 9, or about 1 to about 8, or about 1 to about 7, or about 1 to about 6, or about 1 to about 5, or about 1 to about 4, or about 1.4 to about 5, or about 1.5 to about 3.5. For example, the molecular weight polydispersity index can be about 1, 1.1, 1.2, 1.3, 1.4, 1.5, 1.6, 1.7, 1.8, 1.9, 2.0, 2.5, 3.0, 3.5, 4.0, 4.5, or 5.0.

In embodiments, the compounds of formula (II) can have a degree of branching of 0 to about 0.6, about 0.05 to about 0.5, about 0.01 to about 0.1, about 0.03 to about 0.13, about 0.1 to about 0.45, or about 0.2 to about 0.3. Other suitable values include about 0, 0.01, 0.02, 0.03, 0.04, 0.05, 0.06, 0.07, 0.08, 0.09, 0.1, 0.15, 0.2, 0.25 0.3, 0.35, 0.4, 0.45, 0.5, 0.55, and 0.6.

The crosslinking metal-oxide precursor comprises molecules and polymers that are substantially water immiscible (i.e. oil-soluble) and that are fully hydrolysable such that upon undergoing hydrolysis and condensation reactions, they will form a crosslinking metal-oxide. crosslinking metal-oxide comprise oxides of metals selected from the group of block-d elements (or transition metals) and the group of f-block elements (or inner transition metals).

The crosslinking metal-oxide precursor comprise compounds of formula (III):

[MₐL_{b}X_{c}] Formula (III)
Where each M independently includes d-block and f-block metals,
   Each L independently includes mono- bi- or tri-dentate chelating ligands,
   Each X independently includes alkoxide ligands or halide ligand.
   (a) is an integer from 1 to 3,
   (b) is an integer from 1 to 6×(a)
   (c) is an integer from 0 to 4×(a).

Monodentate chelating ligands include compounds that are bound to the metal center via a single coordination covalent bond. Non-limiting examples are compounds bearing a carbonyl group and an amino group.

Bidentate chelating ligands include compounds that have two separate atoms of the same molecule bound to the metal center via a coordination covalent bond. Non-limiting examples of bidentate chelating ligands are the groups of 1,3- and 1,4-dicarbonyles.

Tridentate chelating ligands include compounds that have three separate atoms of the same molecule bound to the metal center via a coordination covalent bond. Non-limiting examples include diethylene triamine and citric acid and its de-protonated derivatives.

Alkoxide ligands include compounds of formula OR, wherein the oxygen atom is bound to the metal center via a coordination covalent bond. Non-limiting examples are primary alcohols.

Halide ligands include ions from the group of halogens, wherein the halogen atom is bound to the metal center via a coordination covalent bond.

Non-limiting examples of suitable metal centers are Titanium, Zirconium, Hafnium, Zinc, Iron, Copper, Silver, Gold, Chromium, Cobalt, Nickel, Vanadium or Molybdenum.

In embodiments, the metal center is selected from Titanium, Zirconium or their mixture.

In embodiments, the monodentate ligand comprises aldehydes, esters, lactones, secondary amines, nitriles, alkenes and linear alcohols with 10 or more carbon atoms. In a preferred embodiment, the monodentate ligand comprises esters, nitriles, alkenes, lactones and aldehydes bearing 9 or less carbon atoms. In another more preferred embodiment, the monodentate ligand comprises esters, nitriles and alkenes.

### Nanoparticle Layer

In embodiments, the first shell component can further include a nanoparticle layer. The nanoparticle layer can be one or more of SiO₂, TiO₂, Al₂O₃, ZrO₂, ZnO₂. In preferred embodiments the nanoparticle layer can include SiO₂.

The nanoparticles can have an average diameter of about 1 nm to about 500 nm, about 1 nm to 300 nm, about 1 nm to 200 nm, about 5 nm to about 100 nm, about 10 nm to about 100 nm, and about 30 nm to about 100 nm. For example, in embodiments, the nanoparticles can have an average diameter of about 1 nm, 5 nm, 10 nm, 20 nm, 30 nm, 40 nm, 50 nm, 60 nm, 70 nm, 80 nm, 90 nm, 100 nm, 150 nm, 200 nm, 250 nm, 300 nm.

The nanoparticles can be spherical, disk-like or irregular. Any nanoparticle shape is contemplated herein. In embodiments, the nanoparticles can include a surface modification such as but not limited to linear or branched C₁ to C₂₀ alkyl groups, surface amino groups, surface methacrylo groups, surface halogens, or surface thiols. These surface modifications are such that the nanoparticle surface can have covalently bound organic molecules on it.

### Second Shell Component

In embodiments, the capsules can include a second shell component. The second shell component substantially surrounds the first shell component. The second shell component comprises an inorganic compound. In embodiments, the second shell component can provide further stability to the capsules and decrease the permeability of the capsules. Without intending to be bound by theory, it is believed that the second shell component can further contribute to improved performance of the capsules, for example, reducing shell permeability and diffusion of the benefit agent during storage.

In embodiments, the second shell component can include one or more of a metal oxide, a semi-metal oxide, a mineral, and a metal. In embodiments, the second shell component can include one or more of SiO₂, TiO₂, Al₂O₃, ZrO₂, ZnO₂, CaCO₃, Ca₂SiO₄, Fe₂O₃, Fe₃O₄, clay, gold, iron, silver, nickel, and copper. In embodiments, the second shell component can be silica. In embodiments, the second shell component can be silica formed from mineralized sodium silicate.

In embodiments, the second shell component can include silica formed from mineralized sodium silicate. In embodiments, formation of a second shell component comprising silica can create a denser capsule shell due to the deposition of silica within the pores of the first shell component. FIG. 10B illustrates an embodiment of a shell having a second shell component.

In embodiments of the method, the second shell component can be formed by admixing capsules having the first shell component with a solution of second shell component precursor. The solution of second shell component precursor can include a water soluble or oil soluble second shell component precursor. In embodiments, the second shell component precursor can be one or more of a compound of formula (I) as defined above, tetraethoxysilane (TEOS), tetramethoxysilane (TMOS), tetrabutoxysilane (TBOS), triethoxymethylsilane (TEMS), diethoxy-dimethylsilane (DEDMS), trimethylethoxysilane (TMES), and tetraacetoxysilane (TAcS). In embodiments, the second shell component precursor can also include one or more of silane monomers of type Si(YR)₄₋ₙ Rₙ wherein YR is a hydrolysable group and R is a non-hydrolysable group. Examples of such monomers are given earlier in this paragraph, and these are not meant to be limiting the scope of monomers that can be used. In embodiments, the second shell component precursor can include salts of silicate, titanate, aluminate, zirconate and/or zincate. In embodiments, the second shell component precursor can include carbonate and calcium salts. In embodiments, the second shell component precursor can include salts of iron, silver, copper, nickel, and/or gold. In embodiments, the second shell component precursor can include zinc, zirconium, Silicon, titanium, and/or aluminum alkoxides. In embodiments, the second shell component precursor can include one or more of silicate salt solutions such as sodium silicates, silicon tetralkoxide solutions, iron sulfate salt and iron nitrate salt, titanium alkoxides solutions, aluminum trialkoxide solutions, zinc dialkoxide solutions, zirconium alkoxide solutions, calcium salt solution, carbonate salt solution. In certain embodiments, a second shell component comprising CaCO₃ can be obtained from a combined use of Calcium salts and Carbonate salts. In other embodiments, a second shell component comprising CaCO₃ can be obtained from Calcium salts without addition of carbonate salts, via in-situ generation of carbonate ions from CO₂.

The second shell component precursor can include any suitable combination of any of the foregoing listed compounds.

In embodiments, the solution of second shell component precursor can be added dropwise to the capsules. In embodiments, the solution of second shell component precursor and the capsules can be mixed together for about 1 hour to about 24 hours, or about 1 hour to about 12 hours, or about 1 hour to about 5 hours.

For example, the solution of second shell component precursor and the capsules can be mixed together for about 1 hour, 2 hours, 3 hours, 4 hours, or 5 hours. In embodiments, the solution of second shell component precursor and the capsules can be mixed together at room temperature or at elevated temperatures, such as 30° C. to 60° C., 40° C. to 70° C., 40° C. to 100° C. For example, the solution of second shell component precursor and the capsules can be mixed together at a temperature of room temperature, 30° C., 35° C., 40° C., 45° C., 50° C., 55° C., 60° C., 65° C., 70° C., 75° C., 80° C., 90° C., or 100° C.

In embodiments, the solution of second shell component precursor can include the second shell component precursor in an amount of about 1 wt % to about 50 wt % based on the total weight of the solution of second shell component precursor, or about 1 wt % to about 40 wt %, or about 1 wt % to about 30 wt %, or about 1 wt % to about 20 wt %, or about 5 wt % to about 20 wt %. For example, the solution of second shell component precursor can include the second shell component precursor in an amount of about 1 wt %, 5 wt %, 10 wt %, 20 wt %, 30 wt %, 40 wt %, or 50 wt % based on the total weight of the solution of second shell component precursor.

In embodiment, capsules can be admixed with the solution of second shell component precursor in the presence of an acid. In embodiments, it can be a weak acid such as HF and acetic acid. In embodiments, the acid can be a strong acid. In embodiments, the strong acid can include one or more of HCl, HNO₃, H₂SO₄, HBr, HI, HClO₄, and HClO₃. In embodiments, the acid can include HCl. In embodiments, the concentration of the acid in continuous solution can be about 0.01 M to about 5 M, or about 0.1 M to about 5 M, or about 0.1 M to about 2 M, or about 0.1 M to about 1 M. For example, the concentration of the acid in the solution of second shell component precursor can be about 0.1 M, 0.2 M, 0.3 M, 0.4 M, 0.5 M, 1 M, 1.5 M, 2 M, 3 M, 4 M, or 5 M.

In embodiments, the capsules can be admixed with a solution of second shell component precursor in the presence of a base. In embodiments, the base can be one or more of mineral bases, a hydroxide, such as sodium hydroxide, and ammonia. For example, in embodiments, the base can be about 10⁻⁵ M to 0.01M NaOH, or about 10⁻⁵ M to about 1M ammonia.

In embodiments, the process of forming a second shell component can include a change in pH during the process. For example, the process of forming a second shell component can be initiated at an acidic or neutral pH and then a base can be added during the process to increase the pH. Alternatively, the process of forming a second shell component can be initiated at a basic or neutral pH and then an acid can be added during the process to decrease the pH. Still further, the process of forming a second shell component can be initiated at an acid or neutral pH and an acid can be added during the process to further reduce the pH. Yet further the process of forming a second shell component can be initiated at a basic or neutral pH and a base can be added during the process to further increase the pH. Any suitable pH shifts can be used. Further any suitable combinations of acids and bases can be used at any time in the solution of second shell component precursor to achieve a desired pH. In embodiments, the process of forming a second shell component can include maintaining a stable pH during the process with a maximum deviation of +/−0.5 pH unit. For example, the process of forming a second shell component can be maintained at a basic, acidic or neutral pH. Alternatively, the process of forming a second shell component can be maintained at a specific pH range by controlling the pH using an acid or a base. Any suitable pH range can be used. Further any suitable combinations of acids and bases can be used at any time in the solution of second shell component precursor to keep a stable pH at a desirable range.

### Mixed Oxide Shell

The shell comprises a first shell component, comprising a mixed-oxide shell. The mixed-oxide shell comprises silica and a crosslinking metal-oxide. The crosslinking metal-oxide can include oxides of metals selected from d-block elements (or transition metals) or f-block elements (or inner transition metals). In embodiments, the crosslinking metal oxide is selected from the group of Zirconium, Titanium, or mixtures thereof.

The crosslinking metal-oxide can be homogeneously distributed in the condensed layer of the shell. In preferred embodiments, the mixed-oxide shell is a co-polymer between silica and the crosslinking metal-oxide. In embodiments, the crosslinking metal oxide can be present in an amount based on the total weight of the first shell component of more than 300 ppm. In another embodiment, the crosslinking metal oxide can be present in an amount based on the total weight of the first shell component of less than 1%, preferably less than 0.5%, even more preferably less than 0.3%. In another embodiment, the crosslinking metal oxide can be present in an amount based on the total weight of the shell of about 300 ppm to about 0.3%.

### Capsule

In embodiments described herein, the capsules can have a mean shell thickness of about 10 nm to about 10,000 nm, about 10 nm to about 1000 nm, about 170 nm to 10,000 nm, about 170 nm to about 1000 nm, about 300 nm to about 1000 nm. In embodiments, the shell can have a thickness of about 50 nm to about 1000 nm, about 10 nm to about 200 nm, about 100 nm to about 1000 nm, about 200 nm to about 1000 nm, about 300 nm to about 1000 nm, about 300 nm to about 800 nm, about 300 to about 700 nm, about 300 nm to about 500 nm, or about 300 nm to about 400 nm. For example, the shell thickness can be about 10, 20, 30, 40, 50, 60, 70, 80, 90 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 2000, 3000, 4000, 5000, 6000, 7000, 8000, 9000, or 10,000 nm.

In various embodiments described herein, the capsules can have a mean volume weighted capsule diameter of about 0.1 micrometers to about 300 micrometers, about 0.1 micrometers to about 100 micrometers, about 10 micrometers to about 200 micrometers, about 10 micrometers to about 100 micrometers, about 10 micrometers to about 75 micrometers, about 50 micrometers to about 100 micrometers, or about 10 micrometers to about 50 micrometers. Other suitable mean volume weighted capsule diameter of about 0.1, 0.5, 1, 5, 10, 15, 20, 25, 30, 35, 40, 45, 50, 55, 60, 65, 70, 75, 80, 85, 90, 95, 100, 125, 150, 175, 200, 225, 250, 275, or 300 micrometers.

In embodiments, the capsules herein can have an average fracture strength of at least 0.1 MPa, or at least 0.25 MPa, or about 0.1 MPa to about 10 MPa, or about 0.25 MPa to about 10 MPa, or about 0.1 MPa to about 5 MPa, or about 0.25 MPa to about 5 MPa, or about 0.1 MPa to about 3 MPa, or about 0.25 MPa to about 3 MPa. For example, the average fracture strength can be about 0.1 MPa, 0.2 MPa, 0.25 MPa, 0.3 MPa, 0.4 MPa, 0.5 MPa, 0.6 MPa, 0.7 MPa, 0.8 MPa, 0.9 MPa, 1 MPa, 1.1 MPa, 1.2 MPa, 1.3 MPa, 1.4 MPa, 1.5 MPa, 1.6 MPa, 1.7 MPa, 1.8 MPa, 1.9 MPa, 2 MPa, 3 MPa, 4 MPa, or 5 MPa. Fully inorganic capsules, such as certain embodiments herein, have traditionally had poor fracture strength, whereas herein, the fracture strength of the capsules can be greater than 0.25 MPa providing for improved stability and a triggered release of the benefit agent upon a designated amount of rupture stress.

### Core

In embodiments, the core, whether oil-based or aqueous, can include one or more benefit agents, as well as additional components such as excipients, carriers, diluents, and other agents. In embodiments, the core can be a liquid core. In embodiments, the core can be a gel core. In embodiments, the core can be aqueous and include a water-based or water-soluble benefit agent. In embodiments, the core can be oil-based and can include an oil-based or oil-soluble benefit agent. In embodiments, the core has a melting point of less than or equal to 15° C. In embodiments, the core is a liquid at the temperature at which it is utilized in a formulated product.

In embodiments, the core is liquid at and around room temperature.

### Oil-Based Core

An oil-based core is defined as the oil phase present in the core of a core-shell capsule, originating from the emulsification of a dispersed oil phase in a continuous aqueous phase; the aforementioned oil and aqueous phases being substantially immiscible.

In embodiments, the oil-based core, can include about 1 wt % to 100 wt % benefit agent based on the total weight of the core. In embodiments, the core can include about 25 wt % to 100 wt % benefit agent based on the total weight of the core or about 50 wt % to 100 wt % benefit agent based on the total weight of the core. For example, the core can include a benefit agent based on the total weight of the core of about 55 wt %, 60 wt %, 65 wt %, 70 wt %, 75 wt %, 80 wt %, 85 wt %, 90 wt %, 95 wt %, and 100 wt %. In embodiments, the core can include about 80 wt % to 100 wt % benefit agent based on the total weight of the core. For example, the benefit agent can be 90 wt %, 91 wt %, 92 wt %, 93 wt %, 94 wt %, 96 wt %, 97 wt %, 98 wt %, or 99 wt % of the core based on the total weight of the core.

In embodiments, the oil-soluble and/or oil based benefit agent can include one or more of chromogens and dyes, perfume composition, perfume raw materials, flavors, lubricants, silicone oils, waxes, hydrocarbons, higher fatty acids, essential oils, lipids, skin coolants, vitamins, sunscreens, antioxidants, catalysts, malodor reducing agents, odor-controlling materials, softening agents, insect and moth repelling agents, colorants, pigments, pharmaceuticals, pharmaceutical oils, adhesives, bodying agents, drape and form control agents, smoothness agents, wrinkle control agents, sanitization agents, disinfecting agents, germ control agents, mold control agents, mildew control agents, antiviral agents, drying agents, stain resistance agents, soil release agents, fabric refreshing agents and freshness extending agents, chlorine bleach odor control agents, dye fixatives, color maintenance agents, color restoration/rejuvenation agents, anti-fading agents, anti-abrasion agents, wear resistance agents, fabric integrity agents, anti-wear agents, anti-pilling agents, defoamers, anti-foaming agents, UV protection agents, sun fade inhibitors, anti-allergenic agents, fabric comfort agents, shrinkage resistance agents, stretch resistance agents, stretch recovery agents, skin care agents, and natural actives, dyes, phase change materials, fertilizers, nutrients, and herbicides.

In embodiments, the oil-based core can include fragrance oil.

In embodiments, the oil-based and/or oil-soluble benefit agent can include a perfume or a perfume composition. In embodiments, the perfume composition can include one or more of perfume raw materials, essential oils, malodour reducing agents, and odour controlling agents.

In various embodiments, the perfume composition can include one or more perfume raw materials. In embodiments, the perfume composition can include, by weight based on the total weight of the perfume composition, a combination of or individually (1) about 2.5% to about 30%, or about 5% to about 30%, of perfume raw materials characterized by a log P of less than 3.0 and a boiling point of less than 250° C.; (2) about 5% to about 30%, or about 7% to about 25%, of perfume raw material characterized by a log P of less than or equal to 3.0 and a boiling point greater than or equal to 250° C.; (3) about 35% to about 60%, or about 40% to about 55%, of perfume raw materials characterized by having a log P of greater than 3.0 and a boiling point of less than 250° C.; and (4) about 10% to about 45%, or about 12% to about 40%, of perfume raw materials characterized by having a log P greater than 3.0 and a boiling point greater than 250° C.

In embodiments, the benefit agent can have an average log P of greater than or equal to 1.

### Water-Based Core

A water-based core is defined as the aqueous phase present in the core of a core-shell capsule, originating from the emulsification of a dispersed aqueous phase in a continuous oil phase; the aforementioned oil and aqueous phases being substantially immiscible.

In embodiments, the water-based core can include about 1 wt % to 99 wt % benefit agent based on the total weight of the core. In embodiments, the core can include about 1 wt % to 75 wt % benefit agent based on the total weight of the core or about 1 wt % to 50 wt % benefit agent based on the total weight of the core. For example, the core can include a benefit agent based on the total weight of the core of about 1 wt %, 5 wt %, 10 wt %, 15 wt %, 20 wt %, 25 wt %, 30 wt %, 35 wt %, 40 wt %, 45 wt %, and 50 wt %. In embodiments, the core can include about 1 wt % to 20 wt %, 30 wt % to 50 wt %, or 20 wt % to 40 wt %, benefit agent based on the total weight of the core.

In embodiments, the water-based and/or water soluble benefit agent is one or more of perfume compositions, perfume raw materials, perfume, skin coolants, vitamins, sunscreens, antioxidants, glycerin, bleach encapsulates, chelating agents, antistatic agents, insect and moth repelling agents, colorants, antioxidants, sanitization agents, disinfecting agents, germ control agents, mold control agents, mildew control agents, antiviral agents, drying agents, stain resistance agents, soil release agents, chlorine bleach odor control agents, dye fixatives, dye transfer inhibitors, color maintenance agents, optical brighteners, color restoration/rejuvenation agents, anti-fading agents, whiteness enhancers, anti-abrasion agents, wear resistance agents, fabric integrity agents, anti-wear agents, anti-pilling agents, defoamers, anti-foaming agents, UV protection agents, sun fade inhibitors, anti-allergenic agents, enzymes, water proofing agents, fabric comfort agents, shrinkage resistance agents, stretch resistance agents, stretch recovery agents, skin care agents, and natural actives, antibacterial actives, antiperspirant actives, cationic polymers, dyes, metal catalysts, non-metal catalysts, activators, pre-formed peroxy carboxylic acids, diacyl peroxides, hydrogen peroxide sources, and enzymes.

In embodiments, the water-based and/or water soluble benefit agent can include one or more metal catalysts. In embodiments, the metal catalyst can include one or more of dichloro-1,4-diethyl-1,4,8,11-tetraaazabicyclo[6.6.2]hexadecane manganese(II); and dichloro-1,4-dimethyl-1,4,8,11-tetraaazabicyclo[6.6.2]hexadecane manganese(II). In embodiments, the non-metal catalyst can include one or more of 2-[3-[(2-bexyldodecyl)oxy]-2-(sulfooxy)propyl]-3,4-dihydroisoquinolinium, inner salt: 3,4-dihydro-2-[3-[(2-pentylundecyl)oxy]-2-(sulfooxy)propyl]isoquinolinium, inner salt; 2-[3-[(2-butyldecyl)oxy]-2-(sulfooxy)propyl]-3,4-dihydroisoquinolinium, inner salt: 3,4-dihydro-2-[3-(octadecyloxy)-2-(sulfooxy)propyl]isoquinolinium, inner salt; 2-[3-(hexadecyloxy)-2-(sulfooxy)propyl]-3,4-dihydroisoquinolinium, inner salt; 3,4-dihydro-2-[2-(sulfooxy)-3-(tetradecyloxy)propyl]isoquinolinium, inner salt; 2-[3-(dodecyloxy)-2-(sulfooxy)propyl]-3,4-dihydroisoquinolinium, inner salt; 2-[3-[(3-hexyldecyl)oxy]-2-(sulfooxy)propyl]-3,4-dihydroisoquinoliinium, inner salt; 3,4-dihydro-2-[3-[(2-pentylnonyl)oxy]-2-(sulfooxy)propyl]isoquinolinium, inner salt; 3,4-dihydro-2-[3-[(2-propylheptyl)oxy]-2-(sulfooxy)propyl]isoquinolinium, inner salt: 2-[3-[(2-butyloctyl)oxy]-2-(sulfooxy)propyl]-3,4-dihydroisoquinolinium, inner salt; 2-[3-(decyloxy)-2-(sulfooxy)propyl]-3,4-dihydroisoquinolinium, inner salt; 3,4-dihydro-2-[3-(octyioxy)-2-(sulfooxy)propyl]isoquinolinium, inner salt; and 2-[3(2-ethylhexyl)oxy]-2-(sulfooxy)propyl]-3,4-dihydroisoquinolinium, inner salt.

In embodiments, the water-based and/or water soluble benefit agent can include one or more activators. In embodiments, the activator can include one or more of tetraacetyl ethylene dianine (TAED); benzoylcaprolactam (BzCL); 4-nitrobenzoylcaprolactam; 3-chlorobenzoylcaprolactam; benzoyloxybenzenesuilplonate (BOBS); nonanoyloxybenzene, sulphonate (NOBS); phenyl benzoate (PhBz); decanovloxybenzenesulphonate (C₁₀-OBS); benzoylvalerolactam (BZVL); octanoyloxybenzenesulphonate (C₈-OBS); pethydrolyzabie esters; 4-[N-(nonaoyl) amino hexanoyloxy]-benzene sulfonate sodium salt (NtACA-OBS); dodecanoyloxybenzenesulphonate (LOBS or C₁₂—OBS); 10-undecenoyioxybenzenesuilfonate (IUjDOBS or C₁₁-OBS with unsaturation in the 10 position); decanoyloxybenzoic acid (DOBA); (6-oclanamidocaproyl)oxybenzenesulfonate; (6-nonanamidocaproyl) oxybenzenesulfonate; and (6-decanamidocaproyl)oxybenzenesulfonate.

In embodiments, the water-based and/or water soluble benefit agent can include one or more preformed peroxy carboxylic acids. In embodiments, the peroxy carboxylic acids can include one or more of peroxymonosulfuiric acids; perimidic acids; percabonic acids; percarboxilic acids and salts of said acids; phthalimidoperoxyhexanoic acid; amidoperoxyacids; 1,12-diperoxydodecanedioic acid; and monoperoxyphthalic acid (magnesium salt hexahydrate), wherein said amidoperoxyacids may include N,N′-terephthaloyl-di(6-aminocaproic acid), a monononylamide of either peroxysuccinic acid (NAPSA) or of peroxyadipic acid (NAPAA), orN-nonanoylaminoperoxycaproic acid (NAPCA).

In embodiments, the water-based and/or water soluble benefit agent can include one or more diacyl peroxide. In embodiments, the diacyl peroxide can include one or more of dinonanoyl peroxide, didecanoyl peroxide, diundecanoyl peroxide, dilauroyl peroxide, and dibenzoyl peroxide, di-(3,5,5-methyl hexanoyl) peroxide, wherein said diacyl peroxide can be clatharated.

In embodiments, the water-based and/or water soluble benefit agent can include one or more hydrogen peroxide. In embodiments, hydrogen peroxide source can include one or more of a perborate, a percarbonate a peroxyhydrate, a peroxide, a persulfate and mixtures thereof, in one aspect said hydrogen peroxide source may comprise sodium perborate, in one aspect said sodium perborate may comprise a mono- or tetra-hydrate, sodium pyrophosphate peroxyhydrate, urea peroxyhydrate, trisodium phosphate peroxyhydrate, and sodium peroxide.

In embodiments, the water-based and/or water soluble benefit agent can include one or more enzymes. In embodiment, the enzyme can include one or more of peroxidases, proteases, lipases, phospholipases, cellulases, cellobiohydrolases, cellobiose dehydrogenases, esterases, cutinases, pectinases, mannanases, pectate lyases, keratinases, reductases, oxidases, phenoloxidases, lipoxygenases, ligninases, pullulanases, tannases, pentosanases, glucanases, arabinosidases, hyaluronidase, chondroitinase, laccases, amylases, and dnases.

In embodiments, the water-based and/or water-soluble benefit agent can include a perfume or a perfume composition. In embodiments, the perfume composition can include one or more of perfume raw materials, essential oils, malodour reducing agents, and odour controlling agents.

In various embodiments, the perfume composition can include one or more perfume raw materials. In embodiments, the perfume composition can include, by weight based on the total weight of the perfume composition, a combination of or individually (1) about 35% to about 60%, or about 40% to about 55%, of first perfume raw materials characterized by a log P of less than 1.5 and a boiling point of less than 250° C.; (2) about 10% to about 45%, or about 12% to about 40%, of second perfume raw materials characterized by a log P of less than or equal to 1.5 and a boiling point greater than or equal to 250° C.; (3) about 2.5% to about 30%, or about 5% to about 30%, of third perfume raw materials characterized by having a log P of greater than 1.5 and a boiling point of less than 250° C.; and (4) about 5% to about 30%, or about 7% to about 25%, of fourth perfume raw materials characterized by having a log P greater than 1.5 and a boiling point greater than 250° C. In embodiments, the benefit agent can have an average log P less than or equal to 1.

### Method of Making Capsules

### Method for Making Oil-Based Core

In embodiments of the method of making capsules having an oil-based core, the oil phase can include an oil-based and/or oil-soluble benefit agent and a precursor mixture. The precursor mixture is defined earlier in the specifications.

In embodiments, the precursor mixture is present in an amount of about 1 wt % to about 50 wt % based on the total weight of the oil phase. Other suitable amounts include about 1 wt % to about 15 wt %, about 5 wt % to about 30 wt %, about 10 wt % to about 20 wt %, about 15 wt % to about 40 wt %, about 25 wt % to about 45 wt %, or about 15 wt % to about 50 wt %, based on the total weight of the oil phase. For example, the oil phase can include, based on the total weight of the oil phase, about 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, or 50 wt %.

In embodiments, the oil phase, prior to emulsification, can include about 10 wt % to about 99 wt % benefit agent based on the total weight of the oil phase, or about 20 wt % to about 99 wt %, about 40 wt % to about 99 wt %, or about 50 wt % to about 99 wt %, or about 50 wt % to about 90 wt %. For example, the benefit agent can be present in an amount based on the total weight of the oil phase of about 10 wt %, 20 wt %, 30 wt %, 40 wt %, 50 wt %, 60 wt %, 70 wt %, 80 wt %, or 90 wt %.

The oil phase can further include one or more oil-soluble core-modifiers. For example, an oil-soluble core modifier can be one or more of partitioning modifier and/or a density modifier. In embodiments, the partitioning modifier can include oil soluble materials that have a log P greater than about 1, or greater than about 2, or greater than about 3, or greater than about 4, or greater than about 5, or greater than about 6, or greater than about 7, or greater than about 8, or greater than about 9, or greater than about 10, or greater than about 11. In embodiments, the partitioning modifier can include oil soluble materials with a density of greater than or equal to 1 gram per cubic centimeter. In embodiments, the partitioning modifier can include one or more of a mono-ester, di-ester and tri-esters of C₄-C₂₄ fatty acids and glycerine; fatty acid esters of polyglycerol oligomers; polyalphaolefins; silicone oil; crosslinked silicones comprising polyether substituted structural units and acrylate crosslinks; polyglycerol ether silicone crosspolymers; alkyl substituted cellulose; hydroxypropyl cellulose; fatty esters of acrylic or methacrylic acid that have side chain crystallizing groups; copolymers of ethylene, including ethylene and vinyl acetate, ethylene and vinyl alcohol, ethylene/acrylic elastomers; acetyl caryophyllene, hexarose, butyl oleate, hydrogenated castor oil, sucrose benzoate, dodecanoic acid, palmitic acid, stearic acid, tetradecanol, hexadecanol, 1-octanediol, isopropyl myristate, castor oil, mineral oil, isoparaffin, caprylic triglyceride, soybean oil, vegetable oil, brominated vegetable oil, bromoheptane, sucrose octaacetate, geranyl palmitate, acetylcaryophyllene, sucrose benzoate, butyl oleate, silicones, polydimethylsiloxane, vitamin E, decamethylcyclopentasiloxane, dodecamethylcyclohxasiloxane, sucrose soyate, sucrose stearate, sucrose soyanate, lauryl alcohol, 1-tetradecanol, 1-hexadecanol, cetyl alcohol, 1-octadecanol, 1-docosanol, 2-octyl-1-dodecanol, perfume oils, in one aspect perfume oils having a log P>5, in one aspect said perfume oils may be selected from the group consisting of: Octadecanoic acid, octadecyl ester; Tetracosane, 2,6,10,15,19,23-hexamethyl-; Octadecanoic acid, diester dissolved in 1,2,3-propanetriol; Isotridecane, 1,1′-[(3,7-dimethyl-6-octenylidene)bis(oxy)]bis-; Tetradecanoic acid, octadecyl ester; 2,6,10,14,18,22-Tetracosahexaene, 2,6,10,15,19,23-hexamethyl-, (all-E)-; Tricosane; Docosane; Hexadecanoic acid, dodecyl ester; 1,2-Benzenedicarboxylic acid, didodecyl ester; Decanoic acid, 1,2,3-propanetriyl ester; 1-Undecene, 11,11-bis[(3,7-dimethyl-6-octenyl)oxy]-; Heneicosane; Benzene, [2-[bis[(3,7-dimethyl-2,6-octadienyl)oxy]methyl]-1-; 1-Undecene, 11,11-bis[(3,7-dimethyl-2,6-octadienyl)oxy]-; Benzene, [2-[bis[(1-ethenyl-1,5-dimethyl-4-hexenyl)oxy]methyl]-1-; Dodecanoic acid, tetradecyl ester; 2H-1-Benzopyran-6-ol, 3,4-dihydro-2,5,7,8-tetramethyl-2-(4,8,12-trimethyltridecyl)-, [2R-[2R*(4R*,8R*)]]—; Octanoic acid, octadecyl ester; Eicosane; 2H-1-Benzopyran-6-ol, 3,4-dihydro-2,5,8-trimethyl-2-(4,8,12-trimethyltridecyl)-, [2R*(4R*,8R*)]—; 2-Naphthalenol, 1-[6-(2,2-dimethyl-6-methylenecyclohexyl)-4-methyl-3-hexenyl]decahydro-2,5,5,8a-tetramethyl-, [1R-[1.alpha.[E(S*)],2.beta.,4a.beta.,8a.alpha.]]-; 2H-1-Benzopyran-6-ol, 3,4-dihydro-2,7,8-trimethyl-2-(4,8,12-trimethyltridecyl)-, [2R-[2R*(4R*,8R*)]]—; Heptanoic acid, octadecyl ester; Nonadecane; 2,4,6,8,10,12,14,16-Heptadecaoctaenal, 2,6,11,15-tetramethyl-17-(2,6,6-trimethyl-1-cyclohexen-1-yl)-, (2E,4E,6E,8E,10E,12E,14E,16E)-; 2H-1-Benzopyran-6-ol, 3,4-dihydro-2,8-dimethyl-2-(4,8,12-trimethyltridecyl)-, [2R-[2R*(4R*,8R*)]]—; Hexadecanoic acid, 2-ethylhexyl ester; 1,2-Benzenedicarboxylic acid, didecyl ester; Octadecane; Benzoic acid, 2-[[2-(phenylmethylene)octylidene]amino]-,1-ethenyl-1,5-dimethyl-4-hexenyl ester; Octadecanoic acid, 3-methylbutyl ester; Decanoic acid, ester with 1,2,3-propanetriol octanoate; Heptadecane; 1-Hexadecene, 7,11,15-trimethyl-3-methylene-; Dodecanoic acid, decyl ester; Octadecanoic acid, butyl ester; Decanedioic acid, bis(2-ethylhexyl) ester; Benzene, [2,2-bis[(3,7-dimethyl-6-octenyl)oxy]ethyl]-; Benzene, [2,2-bis[(3,7-dimethyl-2,6-octadienyl)oxy]ethyl]-; 9-Octadecenoic acid (Z)—, butyl ester; Octanoic acid, 1,2,3-propanetriyl ester; Hexadecane; Cyclohexene, 4-(5-methyl-1-methylene-4-hexenyl)-1-(4-methyl-3-pentenyl)-; 2-Hexadecen-1-ol, 3,7,11,15-tetramethyl-, acetate, [R—[R*,R*-(E)]]-; Hexadecanoic acid, butyl ester; Octadecanoic acid, ethyl ester; 1-Dodecanol, 2-octyl-; Pentadecane; Tetradecanoic acid, hexyl ester; Decanoic acid, decyl ester; Acetic acid, octadecyl ester; Hexadecanoic acid, 2-methylpropyl ester; 9-Octadecenoic acid (Z)—, ethyl ester; Heptadecanoic acid, ethyl ester; Octadecanoic acid, methyl ester; Tetradecane; Tetradecanoic acid, 3-methylbutyl ester; 2-Hexadecen-1-ol, 3,7,11,15-tetramethyl-, [R—[R*,R*-(E)]]-; 2-Hexadecen-1-ol, 3,7,11,15-tetramethyl-; Hexadecanoic acid, 1-methylethyl ester; 1H-Indole, 1,1′-(3,7-dimethyl-6-octenylidene)bis-; Octadecanoic acid; Cyclopentasiloxane, decamethyl-; Benzoic acid, 2-[[2-(phenylmethylene)octylidene]amino]-,3-methylbutyl ester; 9,12-Octadecadienoic acid (Z,Z)—, ethyl ester; 1-Octadecanol; Hexanedioic acid, dioctyl ester; 9-Octadecenoic acid (Z)—, methyl ester; Octadecanoic acid, 2-hydroxypropyl ester; Tetradecanoic acid, butyl ester; Dodecanoic acid, hexyl ester; 9,12,15-Octadecatrienoic acid, ethyl ester, (Z,Z,Z)—; Hexadecanoic acid, ethyl ester; 1-Hexadecanol, acetate; 9-Octadecenoic acid (Z)—; Hexanedioic acid, bis(2-ethylhexyl) ester; 1,8,11,14-Heptadecatetraene; 1,8,11,14-Heptadecatetraene; 1,8,11,14-Heptadecatetraene; 9-Octadecen-1-ol, (Z)—; Tetradecanoic acid, 2-methylpropyl ester; Nonanoic acid, 1-methyl-1,2-ethanediyl ester; Tridecane; Naphthalene, decahydro-1,6-dimethyl-4-(1-methylethyl)-, [1S-(1.alpha.,4.alpha.,4a.alpha.,6.alpha.,8a.beta.)]-, didehydro deriv.; 1-Hexadecyn-3-ol, 3,7,11,15-tetramethyl-; 9,12-Octadecadienoic acid (Z,Z)—, methyl ester; 1-Heptadecanol; 6,10,14-Hexadecatrien-3-ol, 3,7,11,15-tetramethyl-; Benzoic acid, 2-[[[4-(4-methyl-3-pentenyl)-3-cyclohexen-1-yl]methylene]amino]-, methyl ester; 9,12-Octadecadienoic acid (Z,Z)—; 2-Nonene, 1,1′-oxybis-; Santalol, benzeneacetate; 10-Undecenoic acid, heptyl ester; 9,12,15-Octadecatrienoic acid, methyl ester, (Z,Z,Z)—; Octadecanoic acid, monoester with 1,2,3-propanetriol; Dodecanoic acid, pentyl ester; Octanoic acid, nonyl ester; Pentadecanoic acid, ethyl ester; Hexadecanoic acid, methyl ester; Dodecanoic acid, 4-methylphenyl ester; Dodecanoic acid, 3-methylbutyl ester; Tetradecanoic acid, 1-methylethyl ester; Hexadecanoic acid; 1-Phenanthrenecarboxylic acid, tetradecahydro-1,4a-dimethyl-7-(1-methylethyl)-, methyl ester, [1R-(1.alpha.,4a.beta.,4b.alpha.,7.beta.,8a.beta.,10a.alpha.)]-; 1-Hexadecanol; Dodecane; 2-Pentadecanone, 6,10,14-trimethyl-; 9-Heptadecanone; 1-Phenanthrenemethanol, 1,2,3,4,4a,4b,5,6,10,10a-decahydro-1,4a-dimethyl-7-(1-methylethyl)-, acetate, [1R-(1.alpha.,4a.beta.,4b.alpha.,10a.alpha.)]-; Isohexadecanol; Dodecanoic acid, 2-methylpropyl ester; Hexadecanenitrile; Octadecanoic acid, 2,3-dihydroxypropyl ester; Isododecane; 1-Phenanthrenemethanol, tetradecahydro-1,4a-dimethyl-7-(1-methylethyl)-; Octanoic acid, 3,7-dimethyl-2,6-octadienyl ester, (E)-; Dodecanoic acid, butyl ester; Tetradecanoic acid, ethyl ester; Butanoic acid, dodecyl ester; Benzoic acid, 2-amino-, decyl ester; Oxacycloheptadecan-2-one; Propanoic acid, 2-methyl-, dodecyl ester; 1H-Indene, octahydro-1,1,2,3,3-pentamethyl-; 1-Phenanthrenecarboxylic acid, 1,2,3,4,4a,4b,5,6,7,8,10,10a-dodecahydro-1,4a-dimethyl-7-(1-methylethyl)-, methyl ester; 9-Octadecenoic acid (Z)—, ester with 1,2,3-propanetriol; 9,12,15-Octadecatrienoic acid, (Z,Z,Z)—; 1,4,8-Cycloundecatriene, 2,6,6,9-tetramethyl-, (E,E,E)-; 1-Phenanthrenemethanol, dodecahydro-1,4a-dimethyl-7-(1-methylethyl)-; Benzoic acid, 3,4,5-trihydroxy-, dodecyl ester; 1H-Indole-1-heptanol, .eta.-1H-indol-1-yl-.alpha.,.alpha.,.epsilon.-; Cyclododecane; 9-Hexadecenoic acid, (Z)—; Benzoic acid, 2-[[2-(phenylmethylene)heptylidene]amino]-, methyl; 9-Octadecenoic acid (Z)—, 2,3-dihydroxypropyl ester; 2-Naphthalenecarboxaldehyde, 5,6,7,8-tetrahydro-3,5,5,6,7,8,8-heptamethyl-, trans-; Octanoic acid, 1-ethenyl-1,5-dimethyl-4-hexenyl ester; or 2-Hexadecanone.

In embodiments, the density modifiers can include one or more of brominated vegetable oil; sucrose octaacetate; bromoheptane; titanium dioxide; zinc oxides; iron oxides; cobalt oxides; nickel oxides; silver oxides; copper oxides; zirconium oxides; silica; silver; zinc; iron; cobalt; nickel; copper; epoxidized soybean oil polyols; 1h-indene, 2,3-dihydro-1,1,3,3,5-pentamethyl-4,6-dinitro-; benzene, (2-bromoethenyl)-; benzeneacetic acid, 2-methoxy-4-(1-propenyl)phenyl ester; ethanone, 1-(2,5-dimethyl-3-thienyl)-; oxiranecarboxylic acid, 3-(4-methoxyphenyl)-, ethyl ester; benzoic acid, 2-[(1-hydroxy-3-phenylbutyl)amino]-methyl ester; 1,3-benzodioxole-5-carboxylic acid, ethyl ester; 1,3-benzodioxole, 5-(2-propenyl)-; benzoic acid, 4-methoxy-; benzenemethanol, .alpha.-(trichloromethyl)-, acetate; phenol, 2-methoxy-4-(2-propenyl)-, formate; phenol, 2-methoxy-4-(2-propenyl)-, benzoate; 2-propen-1-ol, 3-phenyl-, benzoate; benzeneacetic acid, 3-methylphenyl ester; benzene, 1-(1,1-dimethylethyl)-3,4,5-trimethyl-2,6-dinitro-; benzeneacetic acid, 4-methylphenyl ester; benzeneacetic acid, phenylmethyl ester; benzeneacetic acid, (4-methoxyphenyl)methyl ester; 2-propenoic acid, 3-phenyl-, phenylmethyl ester; 2-propenoic acid, 3-phenyl-, 2-phenylethyl ester; benzeneacetic acid, 2-methoxy-4-(2-propenyl)phenyl ester; phenol, 2-(methylthio)-; benzoic acid, 2-[[3-(1,3-benzodioxol-5-yl)-2-methylpropylidene]amino]-, methyl ester; benzoic acid, 2-[[3-(4-methoxyphenyl)-2-methylpropylidene]amino]-,methyl ester; benzoic acid, 3,5-dimethoxy-; benzoic acid, 2-hydroxy-, phenyl ester; benzoic acid, 2-hydroxy-, phenylmethyl ester; benzoic acid, 2-hydroxy-, ethyl ester; benzoic acid, 2-hydroxy-, methyl ester; benzoic acid, 2-amino-, methyl ester; ethanone, 2-hydroxy-1,2-diphenyl-; benzoic acid, 4-hydroxy-, ethyl ester; benzoic acid, phenylmethyl ester; 1,3-benzodioxole, 5-(1-propenyl)-; benzothiazole, 2-methyl-; 5h-dibenzo[a,d]cyclohepten-5-one, 10,11-dihydro-; oxiranecarboxylic acid, 3-phenyl-, ethyl ester; benzoic acid, 4-methoxy-, methyl ester; 2-propenoic acid, 3-phenyl-, 3-phenyl-2-propenyl ester; tricyclo[3.3.1.13,7]decan-2-ol, 4-methyl-8-methylene-; tricyclo[3.3.1.13,7]decan-2-ol, 4-methyl-8-methylene-, acetate; methanone, bis(2,4-dihydroxyphenyl)-; methanone, (2-hydroxy-4-methoxyphenyl)phenyl-; dibenzofuran; benzoic acid, 2-amino-, 2-phenylethyl ester; ethanone, 1-(naphthalenyl)-; furan, 2,2′-[thiobis(methylene)]bis-; 1,2,3-propanetriol, tripropanoate; 2-propenoic acid, 3-phenyl-, (e)-; phenol, 4-ethyl-2,6-dimethoxy-; disulfide, methyl phenyl; benzoic acid, 2-[[(4-methoxyphenyl)methylene]amino]-, methyl ester; 2-propenoic acid, 3-(2-methoxyphenyl)-, (z)-; 8-quinolinol; disulfide, bis(phenylmethyl); 1,2-propanediol, dibenzoate; benzene, 1-bromo-4-ethenyl-; trisulfide, di-2-propenyl; phenol, 2,6-dimethoxy-4-(1-propenyl)-, (e)-; benzene, (2-isothiocyanatoethyl)-; benzoic acid, 2-hydroxy-5-methyl-, methyl ester; 1,2,4-trithiolane, 3,5-dimethyl-; propanoic acid, 2-(methyldithio)-, ethyl ester; benzoic acid, 2-hydroxy-, cyclohexyl ester; benzoic acid, 2-[(1-oxopropyl)amino]-, methyl ester; ethanethioic acid, s-(4,5-dihydro-2-methyl-3-furanyl) ester; benzoic acid, 2-(acetylamino)-, methyl ester; 1,3,5-trithiane, 2,4,6-trimethyl-; benzoic acid, 2-amino-, propyl ester; butanoic acid, 1-naphthalenyl ester; benzoic acid, 2,4-dihydroxy-3-methyl-, methyl ester; trisulfide, methyl 2-propenyl; 2-furanmethanol, benzoate; benzoic acid, 2-hydroxy-5-methyl-, ethyl ester; benzene, (2,2-dichloro-1-methylcyclopropyl)-; 2-thiophenecarboxaldehyde, 5-ethyl-; benzoic acid, [(phenylmethylene)amino]-, methyl ester; spiro[1,3-dithiolo[4,5-b]furan-2,3′(2′h)-furan], hexahydro-2′,3a-dimethyl-; 1,3-benzodioxole, 5-(diethoxymethyl)-; cyclododeca[c]furan, 1,3,3a,4,5,6,7,8,9,10,11,13a-dodecahydro-; benzeneacetic acid, 2-methoxyphenyl ester; 2-benzofurancarboxaldehyde; 1,2,4-trithiane, 3-methyl-; furan, 2,2′-[dithiobis(methylene)]bis-; 1,6-heptadiene-3,5-dione, 1,7-bis(4-hydroxy-3-methoxyphenyl)-, (e,e)-; benzoic acid, 2,4-dihydroxy-3,6-dimethyl-, methyl ester; benzoic acid, 2-hydroxy-4-methoxy-, methyl ester; propanoic acid, 2-methyl-, 1,3-benzodioxol-5-ylmethyl ester; 1,2,4-trithiolane, 3,5-diethyl-; 1,2,4-trithiolane, 3,5-bis(1-methylethyl)-; furan, 2-[(methyldithio)methyl]-; tetrasulfide, dimethyl; benzeneacetaldehyde, .alpha.-(2-furanylmethylene)-; benzoic acid, 3-methoxy-; benzenecarbothioic acid, s-methyl ester; benzoic acid, 2-methoxy-, methyl ester; benzoic acid, 2-hydroxy-, 4-methylphenyl ester; benzoic acid, 2-hydroxy-, propyl ester; 2-propenoic acid, 3-(2-methoxyphenyl)-; 2-propenoic acid, 3-(3-methoxyphenyl)-; benzoic acid, 2-hydroxy-4-methoxy-6-methyl-, ethyl ester; benzaldehyde, 2-hydroxy-5-methyl-; 1,2,3-propanetriol, tribenzoate; benzoic acid, 4-methylphenyl ester; 2-furancarboxylic acid, propyl ester; benzoic acid, 2-hydroxy-, 2-methylphenyl ester; benzoic acid, 4-hydroxy-3-methoxy-, ethyl ester; 2-propenoic acid, 3-phenyl-; benzene, 1,3-dibromo-2-methoxy-4-methyl-5-nitro-; benzene, (isothiocyanatomethyl)-; 2-propenoic acid, 3-(2-furanyl)-, ethyl ester; benzenemethanethiol, 4-methoxy-; 2-thiophenemethanethiol; benzene, 1,1′-[(2-phenylethylidene)bis(oxymethylene)]bis-; phenol, 2,6-dimethoxy-4-(2-propenyl)-; benzoic acid, 2-[(2-phenylethylidene)amino]-, methyl ester; benzenepropanoic acid, .beta.-oxo-, 4-methylphenyl ester; 1h-indole-3-heptanol, .eta.-1h-indol-3-yl-.alpha.,.alpha.,.epsilon.-trimethyl-; benzoic acid, 2-hydroxy-, 3-methyl-2-butenyl ester; 1,3-benzodioxole-5-propanol, alpha.-methyl-, acetate; thiophene, 2,2′-dithiobis-; benzoic acid, 2-hydroxy-; benzaldehyde, 2-hydroxy-4-methyl-; disulfide, methyl phenylmethyl; 2-furancarboxylic acid, 2-phenylethyl ester; benzenethiol, 2-methoxy-; benzoic acid, 2-[[(4-hydroxy-3-methoxyphenyl)methylene]amino]-,methyl ester; ethanol, 2-(4-methylphenoxy)-1-(2-phenylethoxy)-; benzeneacetic acid, 3-phenyl-2-propenyl ester; benzoic acid, 2-amino-, 2-propenyl ester; bicyclo[3.2.1]octan-8-one, 1,5-dimethyl-, oxime; 2-thiophenethiol; phenol, 2-methoxy-4-(1-propenyl)-, formate; benzoic acid, 2-amino-, cyclohexyl ester; phenol, 4-ethenyl-2-methoxy-; benzoic acid, 2-hydroxy-, 2-(1-methylethoxy)ethyl ester; ethanone, 1-[4-(1,1-dimethylethyl)-2,6-dimethyl-3,5-dinitrophenyl]-; benzene, 1-(1,1-dimethylethyl)-3,5-dimethyl-2,4,6-trinitro-; 2-propenoic acid, 3-(4-methoxyphenyl)-; benzene, 1-(1,1-dimethylethyl)-2-methoxy-4-methyl-3,5-dinitro-; 1,2-benzenedicarboxylic acid, diethyl ester; ethanone, 1-(3,4-dihydro-2h-pyrrol-5-yl)-; benzoic acid, 2-(methylamino)-, methyl ester; 2h-1-benzopyran-2-one, 7-ethoxy-4-methyl-; benzoic acid, 2-hydroxy-, 2-phenylethyl ester; benzoic acid, 2-amino-, ethyl ester; 2-propen-1-ol, 3-phenyl-, 2-aminobenzoate; phenol, 4-chloro-3,5-dimethyl-; disulfide, diphenyl; 1-naphthalenol; [1,1′-biphenyl]-2-ol; benzenemethanol, alpha.-phenyl-; 2-naphthalenethiol; ethanone, 1-(2-naphthalenyl)-; phenol, 2-methoxy-4-(1-propenyl)-, acetate; 2-naphthalenol, benzoate; benzoic acid, phenyl ester; pyridine, 2-[3-(2-chlorophenyl)propyl]-; benzoic acid, 4-hydroxy-, propyl ester; ethanone, 1-(1-naphthalenyl)-; propanoic acid, 3-[(2-furanylmethyl)thio]-, ethyl ester; 2-propen-1-one, 1,3-diphenyl-; 3-pyridinecarboxylic acid, phenylmethyl ester; benzoic acid, 2-phenylethyl ester; piperidine, 1-[5-(1,3-benzodioxol-5-yl)-1-oxo-2,4-pentadienyl]-,(e,e)-; or benzothiazole.

In embodiments of the method of making capsules having an oil-based core, the aqueous phase (continuous phase) can include water, an acid, and nanoparticles. In embodiments, the aqueous phase has a pH of about 1 to about 14 at least at the time of admixing with the oil phase. Other suitable pH include about 1 to about 5, about 2 to about 7, about 6 to about 7, about 1 to about 4, about 3 to about 7, about 7 to 14, about 8 to 10, about 9 to 11, or about 7 to 9. For example, the pH of the aqueous phase can be about 1, 2, 3, 4, 5, 6, 7, 8, 9, 10, 11, 12, 13 or 14.

In embodiments, the acid can be a strong acid. In embodiments, the strong acid can include one or more of HCl, HNO₃, H₂SO₄, HBr, HI, HClO₄, and HClO₃. In embodiments, the acid can include HCl. In embodiments, the concentration of the acid in continuous solution can be about 0.01 M to about 5 M, or about 0.1 M to about 5 M, or about 0.1 M to about 2 M, or about 0.1 M to about 1 M. For example, the concentration of acid in the continuous solution can be about 0.1 M, 0.2 M, 0.3 M, 0.4 M, 0.5 M, 1 M, 1.5 M, 2 M, 3 M, 4 M, or 5 M.

In embodiments, the acid can be a weak acid, such as HF and acetic acid.

In embodiments of the method of making capsules having an oil-based core, the aqueous phase (continuous phase) can include a base. In embodiments, the base can be one or more of mineral bases, a hydroxide, such as sodium hydroxide, and ammonia. For example, in embodiments, the base can be about 10⁻⁵ M to 0.01M NaOH, or about 10⁻⁵ M to about 1M ammonia.

In embodiments of the method of making capsules having an oil-based core, the pH can be varied throughout the process by the addition of an acid and/or a base. For example, the method can be initiated with an aqueous phase at an acidic or neutral pH and then a base can be added during the process to increase the pH. Alternatively, the method can be initiated with an aqueous phase at a basic or neutral pH and then an acid can be added during the process to decrease the pH. Still further, the method can be initiated with an aqueous phase at an acid or neutral pH and an acid can be added during the process to further reduce the pH. Yet further the method can be initiated with an aqueous phase at a basic or neutral pH and a base can be added during the process to further increase the pH. Any suitable pH shifts can be used. Further any suitable combinations of acids and bases can be used at any time in the method to achieve a desired pH.

Any of the nanoparticles described above can be used in the aqueous phase. In embodiments, the nanoparticles can be present in an amount of about 0.01 wt % to about 10 wt % based on the total weight of the aqueous phase. Other suitable amounts include about 0.05 wt % to about 5 wt %, about 1 wt % to about 10 wt %, about 5 wt % to about 8 wt %, about 2 wt % to about 7 wt %, or about 0.1 wt % to about 1 wt %. For example, the nanoparticles can be present in an amount based on the total weight of the aqueous phase of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 wt %.

In embodiments, the method can include admixing the oil phase and the aqueous phase in a ratio of oil phase to aqueous phase of about 1:10 to about 1:1, about 1:9 to about 1:1, about 1:5 to about 1:1, about 1:3 to about 1:1, about 1:5 to about 1:2, about 1:3 to about 1:1.5. Other suitable ratios include about 1:10, 1:9, 1:8, 1:7, 1:6, 1:5, 1:4, 1:3, 1:2, 2:5, 3:5, 1:1.5, or 1:1.

### Water-Based Core Method of Making

In embodiments of the method of making capsules having an aqueous core, the aqueous phase can include an aqueous benefit agent.

In embodiments, the aqueous phase, prior to emulsification, can include about 1 wt % to about 99 wt % benefit agent based on the total weight of the aqueous phase, or about 20 wt % to about 99 wt %, about 40 wt % to about 99 wt %, or about 50 wt % to about 99 wt %, or about 50 wt % to about 90 wt %. For example, the benefit agent can be present in an amount based on the total weight of the aqueous phase of about 1 wt %, 10 wt %, 20 wt %, 30 wt %, 40 wt %, 50 wt %, 60 wt %, 70 wt %, 80 wt %, or 90 wt %.

In embodiments, the aqueous phase can further include one or more core modifiers. For example, an aqueous core modifier can be one or more of a pH modifier, viscosity modifier, ionic strength modifiers, aesthetic modifiers, density modifiers, and gelling agents. In embodiments, the pH modifier may be incorporated to generate the desired pH in the core. In embodiments, the pH modifier can include any alkali or acid known to those skilled in the art of detergent manufacture, for example, among the alkalis: carbonate and hydroxycarbonate salts of alkaline or alkaline-earth metals, e.g., sodium or potassium hydroxide carbonate; oxides and hydroxides of alkaline or alkaline-earth metals, e.g., magnesium oxide, sodium or potassium hydroxide; citrate, fumarate, succinate, tartarate, maleate, ascorbate, silicate of alkaline or alkaline-earth metals, e.g., sodium citrate; among the acids: citric acid, fumaric acid, succinic acid, tartaric acid, malic acid, ascorbic acid, phosphoric acid, hydrochloric acid, sulfuric acid, sulforous acid.

In embodiments, the viscosity modifiers can include nanofibrillated and microfibrillated cellulose, uncoated or coated with a polymeric thickener, of bacterial or vegetable origin; non-polymeric crystalline, hydroxyl functional materials such as a crystallizable glyceride, including hydrogenated castor oil; naturally derived polymeric structurants such as hydroxyethyl cellulose, hydrophobically modified hydroxyethyl cellulose, carboxymethyl cellulose, polysaccharide derivatives. Suitable polysaccharide derivatives include: pectine, alginate, arabinogalactan (gum Arabic), carrageenan, gellan gum, xanthan gum, guar gum. Suitable viscosity modifiers which can be incorporated include synthetic polymeric structurants, e.g., polycarboxylates, polyacrylates, hydrophobically modified ethoxylated urethanes, hydrophobically modified non-ionic polyols; wherein the polycarboxylate polymer may include one or more of a polyacrylate, and polymethacrylate; a copolymer of unsaturated mono- or di-carbonic acid and C₁-C₃₀ alkyl ester of the (meth)acrylic acid.

In embodiments, the ionic strength modifiers can include one or more carboxylic acid, polycarboxylate, phosphate, phosphonate, polyphosphate, polyphosphonate, and borate. In embodiments, the ionic strength modifiers can further include one or more ionic species, such as one or more of oxydisuccinic acid, aconitic acid, citric acid, tartaric acid, malic acid, maleic acid, fumaric acid, succinic acid, sepacic acid, citaconic acid, adipic acid, itaconic acid, dodecanoic acid, acrylic acid homopolymers and copolymers of acrylic acid, maleic acid, calcium, magnesium, iron, manganese, cobalt, copper, and zinc ions.

In embodiments, the aesthetics modifiers can include one or more colorant, such as dyes or pigments and other aesthetic materials. Non-limiting examples of colorants include Rhodamine, Fluorescein, Phathalocyanine, and alumina. In embodiments, the aesthetics modifiers can include non-limiting examples of particles with different shapes and sizes that can include one or more of epoxy coated metalized aluminium polyethylene terephthalate, polyester beads, candelilla beads, silicates and mixtures thereof.

In embodiments, the density modifiers can include one or more of glycerol, mannitol, sugar alcohols, inorganic salts, titanium dioxide, zinc oxides, iron oxides, cobalt oxides, nickel oxides, silver oxides, copper oxides, zirconium oxides, silica, silver, zinc, iron, cobalt, nickel, or copper.

In embodiments, the water soluble gelling agents can include one or more Lecithins, Calcium alginate, Agar, Carrageenan, Processed eucheuma seaweed, Locust bean gum, carob gum, Guar gum, Tragacanth, Acacia gum, gum arabic, Xanthan gum, Karaya gum, Tara gum, Gellan gum, Konjac, Polysorbates, Pectins, Ammonium phosphatides, Sucrose acetate isobutyrate, Glycerol esters of wood resins, Cellulose, Cellulose derivatives and fatty Acids.

In embodiments, the aqueous core can include an enzyme stabilizer. In embodiments, the enzyme stabilizer can include any conventional enzyme stabilizer such as water soluble sources of calcium and/or magnesium ions. In embodiments, the enzyme stabilizer can include one or more of a reversible protease inhibitor, such as a boron compound including borate, 4-formyl phenylboronic acid, phenylboronic acid and derivatives thereof, compounds such as calcium formate, sodium formate and 1,2-propane diol, and diethylene glycol.

In embodiments of methods of making capsules having an aqueous core, the oil phase can include a precursor mixture as described above.

In embodiments, the precursor mixture, present in the oil phase, can be present in an amount of about 1 wt % to about 50 wt % based on the total weight of the aqueous phase (which ultimately forms the core). Other suitable amounts include about 1 wt % to about 15 wt %, about 5 wt % to about 30 wt %, about 10 wt % to about 20 wt %, about 15 wt % to about 40 wt %, about 25 wt % to about 45 wt %, or about 15 wt % to about 50 wt %, based on the total weight of the aqueous phase. For example, the oil phase can include, based on the total weight of the aqueous phase, about 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, or 50 wt %.

In embodiments of method of making capsules having an aqueous core, nanoparticles can be present in one or both of the aqueous phase and the oil phase. In embodiments, the nanoparticles are present only in the aqueous phase. In embodiments, the nanoparticles are present only in the oil phase. In embodiments, the nanoparticles are present in both the oil phase and the aqueous phase.

Any of the nanoparticles described above can be used in the aqueous phase. In embodiments, the nanoparticles can be present in a total amount, whether in one or both of the aqueous and oil phases, of about 0.01 wt % to about 10 wt % based on the total weight of the aqueous phase. Other suitable amounts include about 0.05 wt % to about 5 wt %, about 1 wt % to about 10 wt %, about 5 wt % to about 8 wt %, about 2 wt % to about 7 wt %, or about 0.1 wt % to about 1 wt %. For example, the nanoparticles can be present in an amount based on the total weight of the aqueous phase of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 wt %.

In embodiments, the method includes admixing the oil phase and the aqueous phase in a ratio of about 10:1 to about 1:1, about 9:1 to about 1:1, about 5:1 to about 1:1, about 3:1 to about 1:1, about 5:1 to about 2:1, about 3:1 to about 1.5:1. Other suitable ratios include about 10:1, 9:1, 8:1, 7:1, 6:1, 5:1, 4:1, 3:1, 2:1, 1.5:1 or 1:1.

### Preparing the Oil Phase

In embodiments, the method of making oil-based core capsules or water-based core-capsules involves adding the silica precursor and the crosslinking metal-oxide precursor into a suitable hydrophobic carrier such that they are miscible. In embodiments, the crosslinking metal-oxide precursor can be added as a purified compound directly into the oil phase. In other embodiments, the crosslinking metal-oxide precursor can be formed in-situ in the oil phase via a ligand exchange reaction. For example, the hydrophobic carrier, benefit agent or any other oil soluble molecule contemplated herein can have the ability to bind to a metal center and replacing a pre-existing ligand, thereby forming a new metal-ligand complex.

A non-limiting example is the class of molecules of aldehydes and esters, which upon mixing with a metal alkoxide are capable of replacing the alkoxide ligands and forming a new metal-carbonyl covalent coordination bond, thereby forming a less reactive metal complex.

### Additional Methods Processing

In embodiments, the method can further include washing and drying the capsules after the process of forming the second shell component, using any suitable methods. For example, centrifugation can be used in a washing step. Drying methods are known in the art. One example of drying can be spray drying.

In embodiments, whether making an oil-based core or aqueous core, the emulsion can be cured under conditions to solidify the precursor thereby forming the capsules.

In embodiments, the reaction temperature for curing can be increased in order to increase the rate at which solidified capsules are obtained. The curing is considered complete when the precursor mixture has fully reacted.

In embodiments, the reaction temperature can be between 20° C. and 100° C. under atmospheric pressure. In embodiments, the reaction temperature can be higher than 100° C. by curing under conditions that allow for an increase in pressure.

In embodiments, the pH of curing can be between 0.5 and 14. The pH can be modified during the curing to modify the reaction rate at which capsules are obtained.

### Combinations

A. A population of capsules comprising:
   a core comprising a benefit agent
   a shell surrounding the core
   wherein the shell surrounding the core comprises a substantially inorganic first shell
   wherein the substantially inorganic first shell comprises a condensed layer comprising a condensation product of a mixture of precursors,
   wherein the mixture of precursors comprises a silica precursor and a crosslinking metal-oxide precursor.
B. The population of capsules from paragraph A, wherein the crosslinking metal-oxide precursor comprises at least one compound of formula (III),

[MₐL_{b}X_{c}] (formula (III)
where M is a metal that includes d-block and f-block elements, or combinations thereof;
each L independently includes mono- bi- or tri-dentate chelating ligands, or combinations thereof;
each X independently includes alkoxy ligands or halo ligands, or combinations thereof;
   (a) is an integer from 1 to 3;
   (b) is an integer from 1 to 6×(a); and
   (c) is an integer from 0 to 4×(a).
C. The population of capsules from paragraph B, wherein the mono-dentate ligand comprises aldehydes, esters, lactones, secondary amines, nitriles, alkenes and linear 10 carbon alcohols, or combinations thereof
   preferably esters, nitriles, alkenes, lactones, aldehydes bearing 9 or less carbon atoms, or combinations thereof
   more preferably esters, nitriles and alkenes or combinations thereof.
D. The population of capsules from any one of paragraph A to C, wherein the silica precursor comprises at least one compound of formula (I) or formula (II):

SiY₄ (formula I)
where each Y is independently selected from OH, —OR2, halo,

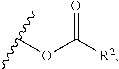

NH2, —NHR2, —N(R2)2, and

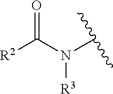

wherein R2 is a C1 to C20 alkyl, C1 to C20 alkylene, C6 to C22 aryl, or a 5-12 membered heteroaryl comprising from 1 to 3 ring heteroatoms selected from O, N, and S;
R3 is a H, C1 to C20 alkyl, C1 to C20 alkylene, C6 to C22 aryl, or a 5-12 membered heteroaryl comprising from 1 to 3 ring heteroatoms selected from O, N, and S;

(SiOzYn)w (formula II)
where z is from 0.5 to 1.6,
each Y is independently selected from
OH, —OR2, halo,

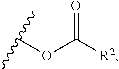

NH2, —NHR2, —N(R2)2, and

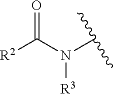

wherein R2 is a C1 to C20 alkyl, C1 to C20 alkylene, C6 to C22 aryl, or a 5-12 membered heteroaryl comprising from 1 to 3 ring heteroatoms selected from O, N, and S;
R3 is a H, C1 to C20 alkyl, C1 to C20 alkylene, C6 to C22 aryl, or a 5-12 membered heteroaryl comprising from 1 to 3 ring heteroatoms selected from O, N, and S;
n is from 0.7 to 4; and
w is from 2 to 2000.
E. The population of capsules from paragraph D, wherein for the compound of formula (II) Y is preferably OR₂, more preferably a OR₂ with R₂ being C1 to C4 alkyl, even more preferably R₂ being C2 alkyl, and wherein its polystyrene equivalent weight averaged molecular weight is between 100 Da and 300.000 Da, preferably between 1000 Da and 10.000 Da.
F. The population of capsules from any one of paragraph B to E, where M includes at least one of Titanium, Zirconium, Hafnium or Iron or combinations thereof, preferably titanium, zirconium or combinations thereof.
G. The population of capsules from anyone of paragraph B to F, where the alkoxy ligands are primary alcohols, preferably methanol, ethanol, isopropanol, butanol.
H. The population of capsules from anyone of paragraph B to F, where the halo ligands are preferably chlorides.
I. A population of capsules comprising:
   a core comprising a benefit agent, and
   a mixed-oxide shell surrounding the core, the mixed-oxide shell comprising:
   silica and a crosslinking metal-oxide,
   wherein the crosslinking metal-oxide is present in an amount of 300 ppm or more based on the total weight of the first shell.
J. The population of capsules from paragraph I, wherein the crosslinking metal oxide is present in an amount of from 300 ppm to 3000 ppm, preferably from 500 ppm to 2500 ppm.
K. The population of capsules from any one of paragraph I to J, wherein the crosslinking metal oxide is an oxide of Titanium, Zirconium, Hafnium or Iron or combinations thereof, preferably of titanium, zirconium, or a combination thereof.
L. The population of capsules from any one of paragraph A to K, wherein the benefit agent comprises a water-based benefit agent, a water soluble benefit agent, an oil based benefit agent, an oil soluble benefit agent, or a combination thereof.
M. The population of capsules from any one of paragraph A to L, wherein the benefit agent is present in the core in an amount of from 10% to 90%, preferably of from 20% to 80% by weight of the capsule.
N. A process of making capsules, preferably making the capsules of any one of paragraph I to M, the process comprising the following steps:
   a. Preparing an oil phase comprising a benefit agent, preferably a benefit agent as disclosed in paragraph L and
   b. Adding a precursor of formula (I) or formula (II) or a mixture thereof, preferably a precursor as disclosed in paragraph D and
   c. Adding a precursor of formula (III), preferably a precursor as disclosed in any one of paragraph B, C or F or adding a metal-alkoxide or metal-halide to form in-situ the compound of formula (III), and
   d. Forming an oil-in-water emulsion between said oil phase and a water phase optionally comprising nanoparticles and optionally comprising an acid, and
   e. Subjecting the oil-in-water emulsion to heat to form capsules via hydrolysis and condensation of the added precursors, and
   f Optionally adding a second-shell component solution to the capsules to form capsules with a second-shell component.
O. The process from paragraph N, wherein the compound of formula (III) is formed in-situ between a metal alkoxide, preferably as disclosed in paragraph G or a metal halide, preferably as disclosed in paragraph H, and the benefit agent from step (a), wherein the mono-dentate ligand comprises preferably esters, nitriles and alkenes.
P. A population of capsules obtainable-by the process from paragraph N or O, wherein the compound of formula (III) preferably comprises a metal selected from Titanium, zirconium and mixtures thereof.
Q. A composition comprising a solvent and a population of capsules according to any combinations of paragraphs A through M, and capsules obtainable by the process of any combinations of the paragraphs N to O.
R. The composition according to paragraph Q, further comprising at least one of a nonionic surfactant, anionic surfactant or cationic surfactant, or a combination thereof.

### Test Methods

### Degree of Branching Method

The degree of branching of the precursors was determined as follows: Degree of branching is measured using (29Si) Nuclear Magnetic Resonance Spectroscopy (NMR).

### Sample Preparation

Each sample is diluted to a 25% solution using deuterated benzene (Benzene-D6 “100%” (D, 99.96% available from Cambridge Isotope Laboratories Inc., Tewksbury, MA, or equivalent). 0.015M Chromium(III) acetylacetonate (99.99% purity, available from Sigma-Aldrich, St. Louis, MO, or equivalent) is added as a paramagnetic relaxation reagent. If glass NMR tubes (Wilmed-LabGlass, Vineland, NJ or equivalent) are used for analysis, a blank sample must also be prepared by filling an NMR tube with the same type of deuterated solvent used to dissolve the samples. The same glass tube must be used to analyze the blank and the sample.

### Sample Analysis

The degree of branching is determined using a Bruker 400 MHz Nuclear Magnetic Resonance Spectroscopy (NMR) instrument, or equivalent. A standard silicon (29Si) method (e.g. from Bruker) is used with default parameter settings with a minimum of 1000 scans and a relaxation time of 30 seconds.

### Sample Processing

The samples are stored and processed using system software appropriate for NMR spectroscopy such as MestReNova version 12.0.4-22023 (available from Mestrelab Research) or equivalent. Phase adjusting and background correction are applied. There is a large, broad, signal present that stretches from −70 to −136 ppm which is the result of using glass NMR tubes as well as glass present in the probe housing. This signal is suppressed by subtracting the spectra of the blank sample from the spectra of the synthesized sample provided that the same tube and the same method parameters are used to analyze the blank and the sample. To further account for any slight differences in data collection, tubes, etc., an area outside of the peaks of interest area should be integrated and normalized to a consistent value. For example, integrate −117 to −115 ppm and set the integration value to 4 for all blanks and samples.

The resulting spectra produces a maximum of five main peak areas. The first peak (Q0) corresponds to unreacted TAOS. The second set of peaks (Q1) corresponds to end groups. The next set of peaks (Q2) correspond to linear groups. The next set of broad peaks (Q3) are semi-dendritic units. The last set of broad peaks (Q4) are dendritic units. When PAOS and PBOS are analyzed, each group falls within a defined ppm range. Representative ranges are described in TABLE 1:

TABLE 1

| Group | Number of Bridging | |
|---|---|---|
| ID | Oxygen per Silicon | ppm Range |
| Q0 | 0 | −80 to −84 |
| Q1 | 1 | −88 to −91 |
| Q2 | 2 | −93 to −98 |
| Q3 | 3 | −100 to −106 |
| Q4 | 4 | −108 to −115 |

The ppm ranges indicated in TABLE 1 above may not apply to all monomers. Other monomers may cause altered chemical shifts, however, proper assignment of Q0-Q4 should not be affected.

Using MestReNova, each group of peaks is integrated, and the degree of branching can be calculated by the following equation:

$$Degree of Branching = 1 / 4 \frac{3 * Q 3 + 4 * Q 4}{Q 1 + Q 2 + Q 3 + Q 4}$$

### Molecular Weight and Polydispersity Index Determination Method

The molecular weight (Polystyrene equivalent Weight Average Molecular Weight (Mw)) and polydispersity index (Mw/Mn) of the condensed layer precursors described herein are determined using Size Exclusion Chromatography with Refractive Index detection. Mn is the number average molecular weight.

### Sample Preparation

Samples are weighed and then diluted with the solvent used in the instrument system to a targeted concentration of 10 mg/mL. For example, weigh 50 mg of polyalkoxysilane into a 5 mL volumetric flask, dissolve and dilute to volume with toluene. After the sample has dissolved in the solvent, it is passed through a 0.45 um nylon filter and loaded into the instrument autosampler.

### Sample Analysis

An HPLC system with autosampler (e.g. Waters 2695 HPLC Separation Module, Waters Corporation, Milford MA, or equivalent) connected to a refractive index detector (e.g. Wyatt 2414 refractive index detector, Santa Barbara, CA, or equivalent) is used for polymer analysis. Separation is performed on three columns, each 7.8 mm I.D.×300 mm in length, packed with 5 μm polystyrene-divinylbenzene media, connected in series, which have molecular weight cutoffs of 1, 10, and 60 kDA, respectively. Suitable columns are the TSKGel G1000HHR, G2000HHR, and G3000HHR columns (available from TOSOH Bioscience, King of Prussia, PA) or equivalent. A 6 mm I.D.×40 mm long 5 μm polystyrene-divinylbenzene guard column (e.g. TSKgel Guardcolumn HHR-L, TOSOH Bioscience, or equivalent) is used to protect the analytical columns. Toluene (HPLC grade or equivalent) is pumped isocratically at 1.0 mL/min, with both the column and detector maintained at 25° C. 100 μL of the prepared sample is injected for analysis. The sample data is stored and processed using software with GPC calculation capability (e.g. ASTRA Version 6.1.7.17 software, available from Wyatt Technologies, Santa Barbara, CA or equivalent.)

The system is calibrated using ten or more narrowly dispersed polystyrene standards (e.g. Standard ReadyCal Set, (e.g. Sigma Aldrich, PN 76552, or equivalent) that have known molecular weights, ranging from about 0.250-70 kDa and using a third order fit for the Mp verses Retention Time Curve.

Using the system software, calculate and report Weight Average Molecular Weight (Mw) and PolyDispersity Index (Mw/Mn).

### Leakage Method

The testing of capsule leakage in liquid solvent compositions is performed as follows via GC/MS techniques.

Homogenized slurry is added and adequately dispersed to a known amount of a solvent based liquid formulation, such that the concentration of the molecule compositions (for example perfume oil) to be evaluated is of 0.25 w % (or between 0.2 w % and 0.3 w %). It is possible for some oils to have a low area in the chromatogram, in which case it can be formulated at a higher level in the solvent based formulation, provided its response on the instrument is adequately calibrated.

The formulated product is stored in a jar or glass container covered with an airtight lid and where the volume of headspace above the liquid is no more than 5× the volume of the liquid itself.

### Sample Preparation

After the desired time of storage, samples of capsules, total oil, and free oil are prepared as follows:
(a) Preparation of capsule sample: 100 microliters of the formulation containing slurry is introduced into the bottom of a Gas Chromatography (GC) vial (see below for specifics of the GC vials and method) and where the GC vials are capped with a crimp cap to yield an airtight milieu, thus obtaining the capsule sample. This step is performed twice to obtain two readings, and the mean of the two values will be used, provided they do not differ too much from each other, in which case the analysis needs to be repeated. The GC vials are then analyzed via Gas Chromatography/Mass Spectrometry (GC/MS), as detailed below.
(b) Preparation of total oil sample: A 1 gram aliquot of the formulation is introduced into a 7 ml cylindrical shape vial of a diameter of 1 cm to 1.5 cm, equipped with a magnetic stirring bar of length no less than the radius of the 7 ml vial, thus ensuring proper mixing in the vial. The 1 gram aliquot in the 7 ml vial is then mixed on a stirring plate for 24 h at 500 rpm, thereby ensuring that the capsules are broken by the grinding action of the stir bar against the bottom of the 7 ml vial. Optical microscopy can be used to verify that no more intact capsules remain. In case such capsules are found, the step is to repeated for an additional 24 h, or until all or almost all capsules are broken. Then, the formulation containing broken capsules is introduced into GC vials in a similar manner as for step (a). This yields total oil samples. It is to be noted that the capsule sample and the total oil sample are not analyzed on the same day, as there is a need to prepare the total oil sample after the leakage sample has been removed from storage. This does not affect (or does not substantially affect) the results.
(c) Preparation of free oil sample: A reference sample (or free oil sample) is prepared by adding and adequately dispersing the same oil as is encapsulated in the capsules to be tested in step (a) into the exact same formulation, at the same level as it is in step (a). Then the free oil formulation is introduced into GC vials in a similar manner as for step (a). This yields reference samples, which must be used when analyzing both the capsule sample and the total oil sample.
   On each day of analysis, the capsule samples or total oil samples should be run in conjunction with the reference sample (or free oil sample).

### Gas Chromatography/Mass Spectrometry GC/MS Method

For each sample, test and reference, aliquots of 100 microliters of sample are transferred to 20 ml headspace vials (Gerstel SPME vial 20 ml, part no. 093640-035-00) and immediately sealed (sealed with Gerstel Crimp caps for SPME, part no. 093640-050-00). Two headspace vials are prepared for each sample. The sealed headspace vials are then allowed to equilibrate. Samples reach equilibrium after 3 hours at room temperature but can be left to sit longer without detriment or change to the results, up until 24 hours after sealing the headspace vial. After equilibrating, the samples are analyzed by GC/MS.

GC/MS analysis are performed by sampling the headspace of each vial via SPME (50/30 μm DVB/Carboxen/PDMS, Sigma-Aldrich part #57329-U), with a vial penetration of 25 millimeters and an extraction time of 1 minute at room temperature. The SPME fiber is subsequently on-line thermally desorbed into the GC injector (270° C., splitless mode, 0.75 mm SPME Inlet liner (Restek, art #23434) or equivalent, 300 seconds desorption time and injector penetration of 43 millimeters). The perfume composition is analyzed by fast GC/MS in full scan mode. Ion extraction of the specific mass for each component is obtained.

### Leakage Calculations

The leakage is calculated as follows, separately for the capsule sample and total oil sample, where “Area” denotes the area under the chromatogram peak corresponding to the oil component of interest:

For each component of the oil, the following formula gives a single oil component leakage:

$$single oil component leakage = \frac{{Area}_{oil component Capsule ( or total oil ) sample}}{{Area}_{oil component reference sample}}$$

Once calculated for all oil components that compose the encapsulated oil for both the total oil sample and the capsule sample, the corrected single oil component leakage can be calculated using the following formula:

$$Corrected single oil component leakage = \frac{single oil component {leakage}_{capsule sample}}{single oil component {leakage}_{total oil sample}}$$

Once the corrected single oil component leakage has been calculated for all oil components, the total leakage can be found by considering the individual leakage values of each oil component and taking into account their relative weight percentage in the full oil composition.

For example, consider an oil which is composed of component A and B, where A represents 70 wt % and B represents 30 wt % of the total composition. For example, component A has a leakage of 20 wt % and component B a leakage of 30 wt %. In order to obtain the total leakage value, the corrected single oil component leakages for A and B are multiplied by their weight percentages (70 wt % and 30 wt % respectively in this example) in the full oil composition, and these new values can then be added to obtain the total leakage:

Total leakage=leakage A*weight % A+leakage B*weight % B=70%*20%+30%*30%=23%.

### Shell Elemental Composition Test Method

Inductively coupled plasma optical emission spectroscopy (ICP-OES) is used to determine the weight fraction of the crosslinking metal oxide in the first shell.

The shell is isolated via a series of centrifugations and solvent washes (suitable solvents are methanol and acetone), which removes the encapsulated core material and any other component that is not part of the first shell from the slurry. The cleaned shell material is then dried using freeze-drying techniques, after which the cleaned and dried first shell materials are ready for elemental analysis.

Samples are prepared in duplicates, and for each replicate 0.02 g of the cleaned and dried shell material is weighted into a 15 ml TFM Ultrawave vial. The sample is then digested an in Ultrawave microwave digestion system (Milestone) with a mixture of 6 ml of nitric acid and 2 ml of hydrofluoric acid. The digestion program consists of a 25 min ramp to 250 C, followed by a 20 min isotherm at 250 C.

After digestion, all samples are to be diluted to 50 ml with deionized water, and adding an internal standard.

A calibration of the elements of interest is to be done with suitable reference standards, inclusive of a suitable internal standard.

The reference standards and samples are then analyzed using an agilent 5110 ICP-OES (or equivalent) instrument, employing multiple wavelengths to demonstrate selectivity and one chosen to analyze the results.

### EXAMPLES

### Silica Precursor Synthesis

To a 22 L 4 neck round bottom flask equipped with mechanical stirrer, addition funnel, thermometer, distillation head, condenser, nitrogen and receiver, 8000 g of tetraethylorthosilicate (98% purity, sigma Aldrich) and 3200 ml of absolute ethanol (Aqua sol) were added. Next, 644.9 g of water were added and stirred for 10 minutes. To this solution, 19.7 g of concentrated HCl (12N or 37.34 wt %, supelco) was added in two portions to control the exotherm.

The reaction vessel was then heated to 78° C. over 30 minutes and stirred for an hour while under reflux in the distillation head. Next, a solution of 7.78 g of sodium hydroxide (pellets, EMD) dissolved in 600 ml of absolute ethanol was slowly added via an addition funnel to control the exotherm such that the reaction temperature remained between 78° C. and 80° C., and further stirred for an additional 10 minutes. Next, the reaction mixture was further heated to observe distillation of ethanol from the vessel, and a constant rate of distillation was maintained until the liquid reached 135° C., after which no more heat was input and the system was refluxing, which was maintained for 1 hour. Next, the product was let to cool to room temperature and 150 g of SIRALOX 80/300 (SASOL Germany GmbH, Z500500) was added to purify the mixture, and the liquids were subsequently filtered over a 1 micron Glass microfibre filters GF/B, (Whatman cat. #1821-110) to give a liquid product. The liquid was split into two equal sized batches, and one half was put under vacuum at 10 mBar at 80° C. until no more distillate was observed. This was finally cooled to room temperature, followed by a final filtration over a 1 micron Glass microfibre filters GF/B, (Whatman cat. #1821-110) to give the final product (2.4 Kg yield).

### Example 1—Silica Capsules with a Different Ligand for Formula (III)

Below is the protocol used for the making of silica capsules. The exact quantities and variations used for each capsule is reported in TABLE 2. During the oil phase preparation, a cyclooctane phase was combined with a silica precursor and a crosslinking metal-oxide precursor. The cyclooctane phase was composed of cyclooctane as the primary component and a different perfume raw material (PRM). The individual PRMs formed complexes with the titanium alkoxide to form compounds of formula (III), whilst the cyclooctane did not interact at all with the titanium alkoxide.

### Aqueous Phase Preparation:

344.75 g of 0.1M HCl (sigma Aldrich) and 5.25 g of aerosil 300 (Evonik) were added into the vessel of an IKA Magic Lab and roughly combined. Next, the dispersion of aerosil 300 in 0.1M HCl was passed 5 times through the 3 rotor-stators (First a coarse, then a medium and then a fine rotor stator) of the instrument at 26000 rpm (for this volume of liquid, 5 passes=1 min and 7 seconds). This resulted in a dispersion of 1.5 wt % of aerosil 300 in 0.1M HCl, ready for use.

### Oil Phase Preparation:

In a beaker equipped with a magnetic stir bar, cyclooctane (CAS #292-64-8, sigma Aldrich), a perfume raw material (PRM), the silica precursor synthetized above, and a titanium alkoxide were combined and mixed until homogeneous.

### Emulsification:

The water phase and the oil phase were added to a 250 ml Polypropylene container from VWR equipped with a screw cap, and the mixture was dispersed via an IKA ultraturrax equipped with an S25N mixing head at 7000 RPM for 2 minutes, to obtain an oil in water emulsion.

### Curing:

The emulsion was kept in the 250 ml polypropylene bottle, and the cap was sealed with Teflon tape to avoid evaporation of volatiles. The emulsion was subsequently left standing in an oven at 90° C. for 16 hours, after which cured capsules were obtained.

### Post-Treatment:

The cured capsules were placed on a lab shaker to ensure a homogeneous distribution of the capsules in the volume of the liquid.

50.3 g of the cured capsule slurry was introduced into a 125 ml Wheaton Cellstir reactor, and deionized water was added to reach a total weight of liquid of 90. The mixture was stirred at 300 rpm whilst adding 2.8 ml of a 10 wt % sodium metasilicate solution (prepared by dissolving anhydrous sodium metasilicate powder purchased from thermos scientific into deionized water, CAS #6834-92-0) via a syringe pump at a flow rate of 80 microliters/min.

Finally, the mixture was centrifuged (2500 rpm for 10 min), and the supernatant liquid was fully removed.

TABLE 2

| | | Cyclooctane | Water | | Titanium | Silica |
|---|---|---|---|---|---|---|
| | | phase | phase | | alkoxide | precursor |
| Example | | quantity | level | | quantity | level |
| ID | PRM name | added (g) | (g) | Titanium alkoxide type added | added (g) | added (g) |
| A | Jasmolactone | 20.0372 | 81.21 | Tetrakis(trimethylsilyloxy) | 0.132 | 13.2 |
| | (CAS# 32764-98-0) | | | titanium (CAS# 15990-66-6) | | |
| B | gamma decalactone | 20.0835 | 80.94 | Tetrakis(trimethylsilyloxy) | 0.142 | 14.18 |
| | (CAS# 706-14-9) | | | titanium (CAS# 15990-66-6) | | |
| C | Decanol | 19.42 | 81.48 | Tetrakis(trimethylsilyloxy) | 0.130 | 13.01 |
| | (CAS# 112-30-1) | | | titanium (CAS# 15990-66-6) | | |
| D | IPM | 19.56 | 80.38 | Tetrakis(trimethylsilyloxy) | 0.135 | 13.54 |
| | (CAS# 110-27-0) | | | titanium (CAS# 15990-66-6) | | |
| E | ethyl decanoate | 19.48 | 80.28 | Tetrakis(trimethylsilyloxy) | 0.137 | 13.69 |
| | (CAS# 110-38-3) | | | titanium (CAS# 15990-66-6) | | |
| F | methyl anthranilate | 19.33 | 80.98 | Tetrakis(trimethylsilyloxy) | 0.136 | 13.57 |
| | (CAS# 134-20-3) | | | titanium (CAS# 15990-66-6) | | |
| G | dimethyl anthranilate | 19.5 | 80.76 | Tetrakis(trimethylsilyloxy) | 0.136 | 13.61 |
| | (CAS# 85-91-6) | | | titanium (CAS# 15990-66-6) | | |
| H | Decanonitrile | 19.45 | 81 | Tetrakis(trimethylsilyloxy) | 0.137 | 13.73 |
| | (CAS# 1975-78-6) | | | titanium (CAS# 15990-66-6) | | |
| I | ethyl 2 methyl | 19.47 | 80.22 | Tetrakis(trimethylsilyloxy) | 0.136 | 13.6 |
| | butyrate (CAS# | | | titanium (CAS# 15990-66-6) | | |
| | 7452-79-1) | | | | | |
| J | Decanal (CAS# | 19.32 | 82.69 | Tetrakis(trimethylsilyloxy) | 0.135 | 13.51 |
| | 112-31-2) | | | titanium (CAS# 15990-66-6) | | |
| K | Hexanal (CAS# | 19.39 | 85.22 | Tetrakis(trimethylsilyloxy) | 0.134 | 13.43 |
| | 66-25-1) | | | titanium (CAS# 15990-66-6) | | |
| L | 1-decene (CAS# | 19.31 | 82.56 | Tetrakis(trimethylsilyloxy) | 0.132 | 13.22 |
| | 872-05-9) | | | titanium (CAS# 15990-66-6) | | |
| M | | 19.62 | 81.58 | Titanium butoxide | 0.108 | 13.34 |
| | | | | (CAS# 5593-70-4) | | |

Except for example ID M from TABLE 2, the cyclooctane phase was composed of 95 w % of cyclooctane, and 5 w % of the PRM. For example ID M, cyclooctane composition was 100%.

### Example 2—Silica Capsule with a Different Quantity of Crosslinking Metal Oxide in the First Shell

Below is the protocol used for the making of silica capsules. The exact quantities and variations used for each capsule is reported in TABLE 3.

### Aqueous Phase Preparation:

378.8 g of 0.1M HCl (sigma Aldrich) and 3.8 g of aerosil 300 (Evonik) were added into the vessel of an IKA Magic Lab and roughly combined. Next, the dispersion of aerosil 300 in 0.1M HCl was passed 5 times through the 3 rotor-stators (First a coarse, then a medium and then a fine rotor stator) of the instrument at 26000 rpm (for this volume of liquid, 5 passes=1 min and 7 seconds). This resulted in a dispersion of 1 wt % of aerosil 300 in 0.1M HCl, ready for use.

### Oil Phase Preparation:

In a beaker equipped with a magnetic stir bar, 79 g of a perfume mixture, 54 g of an equivalent silica precursor as synthetized earlier, and a variable quantity of Zirconium (IV) butoxide (80% in butanol, CAS #1071-76-7, Sigma Aldrich) were combined and mixed until homogeneous. The exact quantities of Zirconium butoxide used are reported in TABLE 3.

### Emulsification:

The water phase was added to a tall 400 ml glass beaker, mounted with an IKA ultraturrax rotor stator equipped with an S25N mixing tool. The oil phase was slowly poured into the water phase over the duration of 30 seconds, whilst mixing with the ultraturrax at 7000 rpm. Once the entirety of the oil phase was added, the mixing was continued for another 2 minutes at 7000 rpm. Afterwards, the emulsion was ready for curing.

### Curing:

The emulsion was introduced into a 500 ml jacketed reactor equipped with a 5 cm diameter mixing blade. The emulsion was left standing for 30 minutes at 30 C. Next, mixing was initiated at 80 rpm for 1 hour and 30 minutes at 30 C, followed by a 1 hour ramp to 90 C. The curing was pursued for another 16 hours at 90 C, after which a cooling ramp of 1 hour lowered the temperature back to room temperature. This yielded cured capsules. A portion of the cured capsules were subjected to a post-treatment, and another portion was used to determine the crosslinking metal oxide content in the first shell.

### Post-Treatment:

125 g of the cured capsules was introduced into a cellstir (Wheaton) reactor of 250 ml mounted on a stirring plate. 3.58 ml of waterglass (sigma Aldrich) was added via a syringe pump at an addition rate of 60 microliters/min, whilst stirring at 350 rpm. After addition of the entire waterglass quantity, the pH was at 5, which was subsequently increased to 7 by the addition of a few drops of 1M NaOH. The obtained capsules were then ready to be used for leakage testing.

TABLE 3

| Example | Added zirconium butoxide |
|---|---|
| ID | (80 vol % in butanol solution) (g) |
| N | 0 |
| O | 0.06 |
| P | 0.15 |
| Q | 0.31 |
| R | 0.46 |
| S | 0.61 |
| T | 0.77 |
| U | 0.92 |
| V | 1.07 |

### Example 3—Leakage of Silica Capsules with Different Ligands for Formula (III)

The capsules from example 1 were formulated into a solvent based liquid formulation (composition in TABLE 4), and aged for one week at room temperature.

TABLE 4

| | WEIGHT PERCENTAGE |
|---|---|
| COMPONENT | IN FORMULATION |
| PROPYLENE GLYCOL (CAS# 57-55-6, | 20.4% |
| SIGMA ALDRICH) | |
| DIPROPYLENE GLYCOL (CAS# | 43.9% |
| 25265-71-8, SIGMA ALDRICH) | |
| DEIONIZED WATER | 35.7% |

The leakage of cyclooctane was measured to give an adequate single variable metric into the effect of each ligand on the porosity of the obtained capsules. A separate calibration curve for the cyclooctane quantification was prepared for each sample. Leakage values below 200 could not be accurately determined and were reported as “<20% leakage”.

The samples were then treated according to the leakage test method described earlier, and the results are reported in TABLE 5 below.

TABLE 5

| Example | | | Cyclooctane |
|---|---|---|---|
| ID | PRM name | Ligand of interest | leakage % |
| A | Jasmolactone | Jasmolactone | 38.6% |
| B | gamma decalactone | gamma decalactone | 21.7% |
| C | Decanol | Decanol | ≤20% |
| D | IPM | IPM | ≤20% |
| E | ethyl decanoate | ethyl decanoate | ≤20% |
| F | methyl anthranilate | methyl anthranilate | 121.4% |
| G | dimethyl anthranilate | dimethyl anthranilate | 41.3% |
| H | decanonitrile | decanonitrile | ≤20% |
| I | ethyl 2 methyl | ethyl 2 methyl | 26.8% |
| | butyrate | butyrate | |
| J | decanal | decanal | 42.4% |
| K | hexanal | hexanal | ≤20% |
| L | 1-decene | 1-decene | ≤20% |
| M | | Butanol | 50.8% |

### Example 4—Impact of Crosslinking Metal Oxide Level in the Shell on Silica Capsule Leakage

The capsules from example 2 were formulated into 2 different liquid formulations:
the same solvent based formulation from example 3 (TABLE 4 for composition), and
a solvent based formulation further comprising a nonionic surfactant (TABLE 6 for composition).

TABLE 6

| | WEIGHT PERCENTAGE |
|---|---|
| COMPONENT | IN FORMULATION |
| ETHANOL (CAS# 64-17-5) | 3% |
| DIETHYLENE GLYCOL (CAS# 111-46-6) | 0.1% |
| DEIONIZED WATER | 88% |
| NONIONIC SURFACTANT | 0.1% |
| OTHER | 8.8% |

The capsules were aged for 1 day at room temperature in the solvent based composition, and 1 week at 35 C in the solvent based formulation further comprising a nonionic surfactant. The leakage after aging was analyzed via the leakage test method reported earlier, in this case for the full encapsulated perfume.

Furthermore, the Zirconium content in the shell was quantified via the shell elemental composition test method reported earlier.

Both leakage results and the Zirconium content are reported in TABLE 7 below.

TABLE 7

| | Zirconium | | Leakage in solvent |
|---|---|---|---|
| | content in | | based formulation |
| Example | the first | Leakage in solvent | further comprising a |
| ID | shell (ppm) | based formulation | nonionic surfactant |
| N | Under limit | 58% | 68% |
| | of detection | | |
| O | 240 | 53% | 44% |
| P | 313 | 40% | 17.50% |
| Q | 509 | 31% | 16% |
| R | 1400 | 35% | 9% |
| S | 1730 | 35% | 8% |
| T | 2150 | 40% | 17% |
| U | 2830 | 53% | 16% |
| V | 3240 | 72% | 26% |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A population of capsules comprising:
a core comprising a benefit agent;
a shell surrounding the core;
wherein the shell surrounding the core comprises a substantially inorganic first shell;
wherein the substantially inorganic first shell comprises a condensed layer comprising a condensation product of a mixture of precursors; and
wherein the mixture of precursors comprises a silica precursor and a crosslinking metal-oxide precursor.

2. The population of capsules from claim 1, wherein the crosslinking metal-oxide precursor comprises at least one compound of formula (III),
[MₐL_{b}X_{c}] (formula (III);
wherein M is a metal that comprises d-block and f-block elements, or combinations thereof;
each L independently comprises mono- bi- or tri-dentate chelating ligands; or combinations thereof;
each X independently comprises alkoxy ligands or halo ligand; or combinations thereof;
(a) is an integer from 1 to 3;
(b) is an integer from 1 to 6×(a); and
(c) is an integer from 0 to 4×(a).

3. The population of capsules from claim 2, wherein the mono-dentate ligand is selected from aldehydes, esters, lactones, secondary amines, nitriles, alkenes and linear 10 carbon alcohols, and combinations thereof.

4. The population of capsules from claim 2, wherein the mono-dentate ligand is selected from esters, nitriles, alkenes, lactones, aldehydes bearing 9 or less carbon atoms, and combinations thereof.

5. The population of capsules from claim 2, wherein the mono-dentate ligand is selected from esters, nitriles, alkenes, and combinations thereof.

6. The population of capsules from claim 1, wherein the silica precursor comprises at least one compound of formula (I) or formula (II):
SiY₄ (formula I)
wherein each Y is independently selected from OH, —OR2, halo, 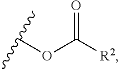
NH2, —NHR2, —N(R2)2, and 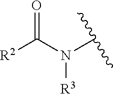
wherein R2 is a C1 to C20 alkyl, C1 to C20 alkylene, C6 to C22 aryl, or a 5-12 membered heteroaryl comprising from 1 to 3 ring heteroatoms selected from O, N, and S; and
wherein R3 is a H, C1 to C20 alkyl, C1 to C20 alkylene, C6 to C22 aryl, or a 5-12 membered heteroaryl comprising from 1 to 3 ring heteroatoms selected from O, N, and S;
(SiOzYn)w (formula II)
wherein z is from 0.5 to 1.6;
each Y is independently selected from
OH, —OR2, halo, 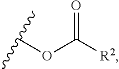
NH2, —NHR2, —N(R2)2, and 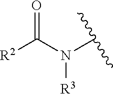
wherein R2 is a C1 to C20 alkyl, C1 to C20 alkylene, C6 to C22 aryl, or a 5-12 membered heteroaryl comprising from 1 to 3 ring heteroatoms selected from O, N, and S;
wherein R3 is a H, C1 to C20 alkyl, C1 to C20 alkylene, C6 to C22 aryl, or a 5-12 membered heteroaryl comprising from 1 to 3 ring heteroatoms selected from O, N, and S;
n is from 0.7 to 4; and
w is from 2 to 2000.

7. The population of capsules from claim 2, where M includes at least one of Titanium, Zirconium, Hafnium or Iron.

8. A population of capsules comprising:
a core comprising a benefit agent, and
a mixed-oxide shell surrounding the core, the mixed-oxide shell comprising:
silica and a crosslinking metal-oxide;
wherein the crosslinking metal-oxide is present in an amount of 300 ppm or more based on the total weight of the first shell.

9. The population of capsules from claim 8, wherein the crosslinking metal oxide is present in an amount of 3000 ppm or less.

10. The population of capsules from claim 8, wherein the crosslinking metal oxide is an oxide of titanium, zirconium, or a mixture thereof.

11. A process of making capsules, the process comprising the following steps:
a. preparing an oil phase comprising a benefit agent, and
b. adding a precursor of formula (I) or formula (II) or a mixture thereof, and
c. adding a precursor of formula (III) or adding a metal-alkoxide or metal-halide to form in-situ the compound of formula (III), and
d. forming an oil-in-water emulsion between said oil phase and a water phase, optionally comprising nanoparticles and optionally comprising an acid, and
e. subjecting the oil-in-water emulsion to heat to form capsules via hydrolysis and condensation of the precursors of the added precursors, and
f. optionally adding a second-shell component solution to the capsules to form capsules with a second-shell component;
wherein Formula (III) is [MₐL_{b}X_{c}] (formula (III);
wherein M is a metal that comprises d-block and f-block elements, or combinations thereof;
each L independently comprises mono- bi- or tri-dentate chelating ligands; or combinations thereof;
each X independently comprises alkoxy ligands or halo ligand; or combinations thereof;
(a) is an integer from 1 to 3;
(b) is an integer from 1 to 6×(a); and
(c) is an integer from 0 to 4×(a);
and formula (I) and formula (II) are as follows:
SiY₄ (formula I)
wherein each Y is independently selected from OH, —OR2, halo, 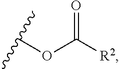
NH2, —NHR2, —N(R2)2, and 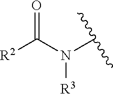
wherein R2 is a C1 to C20 alkyl, C1 to C20 alkylene, C6 to C22 aryl, or a 5-12 membered heteroaryl comprising from 1 to 3 ring heteroatoms selected from O, N, and S; and
wherein R3 is a H, C1 to C20 alkyl, C1 to C20 alkylene, C6 to C22 aryl, or a 5-12 membered heteroaryl comprising from 1 to 3 ring heteroatoms selected from O, N, and S;
(SiOzYn)w (formula II)
wherein z is from 0.5 to 1.6;
each Y is independently selected from
OH, —OR2, halo, 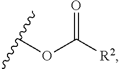
NH2, —NHR2, —N(R2)2, and 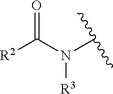
wherein R2 is a C1 to C20 alkyl, C1 to C20 alkylene, C6 to C22 aryl, or a 5-12 membered heteroaryl comprising from 1 to 3 ring heteroatoms selected from O, N, and S;
wherein R3 is a H, C1 to C20 alkyl, C1 to C20 alkylene, C6 to C22 aryl, or a 5-12 membered heteroaryl comprising from 1 to 3 ring heteroatoms selected from O, N, and S;
n is from 0.7 to 4; and
w is from 2 to 2000.

12. The process from claim 11, wherein the compound of formula (III) is formed in-situ between a metal alkoxide or a metal halide, and the benefit agent from step (a) is selected from aldehydes, esters, lactones, secondary amines, nitriles, alkenes, linear 10 carbon alcohols, and combinations thereof.

13. The process of claim 11, wherein the benefit agent from step (a) is selected from esters, nitriles, alkenes, lactones, aldehydes bearing 9 or less carbon atoms, and combinations thereof.

14. The process of claim 11, wherein the benefit agent from step (a) is selected from esters, nitriles, alkenes, and combinations thereof.

15. The process from claim 11, wherein the compound of formula (III) comprises a metal selected from titanium, zirconium and mixtures thereof.
